SEQUENCE LISTING (74 sequences)

SEQ ID NO: 1 - protein, 4 aa, Artificial Sequence
  Peptide-tag
SAYR

SEQ ID NO: 2 - protein, 4 aa, Artificial Sequence
  Peptide-tag
SAYG

SEQ ID NO: 3 - protein, 13 aa, Artificial Sequence
  Tagged peptide
LTGEVVALKSAYG

SEQ ID NO: 4 - protein, 23 aa, Artificial Sequence
  Tagged peptide
LIDWGLAEFYHPAQEYNVRSAYG

SEQ ID NO: 5 - protein, 24 aa, Artificial Sequence
  Tagged peptide
EQSQPCADNAVLSSGLTAARSAYG

SEQ ID NO: 6 - protein, 13 aa, Artificial Sequence
  Tagged peptide
ELIFQETARSAYG

SEQ ID NO: 7 - protein, 13 aa, Artificial Sequence
  Tagged peptide
LAYTFEVQKSAYG

SEQ ID NO: 8 - protein, 14 aa, Artificial Sequence
  Tagged peptide
IISIFSGTEKSAYG

SEQ ID NO: 9 - protein, 24 aa, Artificial Sequence
  Tagged peptide
SVIDPVPAPVGDSHVDGAAKSAYG

SEQ ID NO: 10 - protein, 17 aa, Artificial Sequence
  Tagged peptide
LGINLLGGPLGGKSAYG

SEQ ID NO: 11 - protein, 14 aa, Artificial Sequence
  Tagged peptide
SPEVLLGSARSAYG

SEQ ID NO: 12 - protein, 16 aa, Artificial Sequence
  Tagged peptide
NLDENGLDLLSKSAYG

SEQ ID NO: 13 - protein, 18 aa, Artificial Sequence
  Tagged peptide
LDTETEGVPSTAIRSAYG

SEQ ID NO: 14 - protein, 13 aa, Artificial Sequence
  Tagged peptide
LTGEVVALKSAYG

SEQ ID NO: 15 - protein, 22 aa, Artificial Sequence
  Tagged peptide
TVGALQVLGTEAQSSLLKSAYG

SEQ ID NO: 16 - protein, 18 aa, Artificial Sequence
  Tagged peptide
NLSSNEAISLEEIRSAYG

SEQ ID NO: 17 - protein, 14 aa, Artificial Sequence
  Tagged peptide
HGDLPDIQIKSAYG

SEQ ID NO: 18 - protein, 15 aa, Artificial Sequence
  Tagged peptide
AALSALESFLKSAYG

SEQ ID NO: 19 - protein, 18 aa, Artificial Sequence
  Tagged peptide
SLGPPQGEEDSVPRSAYG

SEQ ID NO: 20 - protein, 13 aa, Artificial Sequence
  Tagged peptide
GQVFDVGPRSAYG

SEQ ID NO: 21 - protein, 13 aa, Artificial Sequence
  Tagged peptide
ELIFEETARSAYG

SEQ ID NO: 22 - protein, 19 aa, Artificial Sequence
  Tagged peptide
LDLDLTADSQPPVFKSAYG

SEQ ID NO: 23 - protein, 21 aa, Artificial Sequence
  Tagged peptide
LSPPYSSPQEFAQDVGRSAYG

SEQ ID NO: 24 - protein, 16 aa, Artificial Sequence
  Tagged peptide
DHQYQFLEDAVRSAYG

SEQ ID NO: 25 - protein, 20 aa, Artificial Sequence
  Tagged peptide
LPNGVFTPDFQEFVNKSAYG

SEQ ID NO: 26 - protein, 14 aa, Artificial Sequence
  Tagged peptide
LGLEDFESLKSAYG

SEQ ID NO: 27 - protein, 23 aa, Artificial Sequence
  Tagged peptide
LIDWGLAEFYHPAQEYNVRSAYG

SEQ ID NO: 28 - protein, 17 aa, Artificial Sequence
  Tagged peptide
HLVSPEALDLLDKSAYG

SEQ ID NO: 29 - protein, 24 aa, Artificial Sequence
  Tagged peptide
EQSQPCADNAVLSSGLTAARSAYG

SEQ ID NO: 30 - protein, 13 aa, Artificial Sequence
  Tagged peptide
ELIFQETARSAYG

SEQ ID NO: 31 - protein, 13 aa, Artificial Sequence
  Tagged peptide
LAYTFEVQKSAYG

SEQ ID NO: 32 - protein, 4 aa, Artificial Sequence
  Peptide-tag
YSGK

SEQ ID NO: 33 - protein, 14 aa, Artificial Sequence
  Tagged peptide
YSGKATHYFIDLNK

SEQ ID NO: 34 - protein, 14 aa, Artificial Sequence
  Tagged peptide
YSGKDTHYFIDLNK

SEQ ID NO: 35 - protein, 14 aa, Artificial Sequence
  Tagged peptide
YSGKETHYFIDLNK

SEQ ID NO: 36 - protein, 14 aa, Artificial Sequence
  Tagged peptide
YSGKFTHYFIDLNK

SEQ ID NO: 37 - protein, 14 aa, Artificial Sequence
  Tagged peptide
YSGKGTHYFIDLNK

SEQ ID NO: 38 - protein, 14 aa, Artificial Sequence
  Tagged peptide
YSGKHTHYFIDLNK

SEQ ID NO: 39 - protein, 14 aa, Artificial Sequence
  Tagged peptide
YSGKITHYFIDLNK

SEQ ID NO: 40 - protein, 14 aa, Artificial Sequence
  Tagged peptide
YSGKKTHYFIDLNK

SEQ ID NO: 41 - protein, 14 aa, Artificial Sequence
  Tagged peptide
YSGKLTHYFIDLNK

SEQ ID NO: 42 - protein, 14 aa, Artificial Sequence
  Tagged peptide
YSGKMTHYFIDLNK

SEQ ID NO: 43 - protein, 14 aa, Artificial Sequence
  Tagged peptide
YSGKNTHYFIDLNK

SEQ ID NO: 44 - protein, 14 aa, Artificial Sequence
  Tagged peptide
YSGKPTHYFIDLNK

SEQ ID NO: 45 - protein, 14 aa, Artificial Sequence
  Tagged peptide
YSGKQTHYFIDLNK

SEQ ID NO: 46 - protein, 14 aa, Artificial Sequence
  Tagged peptide
YSGKRTHYFIDLNK

SEQ ID NO: 47 - protein, 14 aa, Artificial Sequence
  Tagged peptide
YSGKSTHYFIDLNK

SEQ ID NO: 48 - protein, 14 aa, Artificial Sequence
  Tagged peptide
YSGKTTHYFIDLNK

SEQ ID NO: 49 - protein, 14 aa, Artificial Sequence
  Tagged peptide
YSGKVTHYFIDLNK

SEQ ID NO: 50 - protein, 14 aa, Artificial Sequence
  Tagged peptide
YSGKWTHYFIDLNK

SEQ ID NO: 51 - protein, 14 aa, Artificial Sequence
  Tagged peptide
YSGKYTHYFIDLNK

SEQ ID NO: 52 - protein, 14 aa, Artificial Sequence
  Tagged peptide
YSGKAVALSLDDVK

SEQ ID NO: 53 - protein, 14 aa, Artificial Sequence
  Tagged peptide
YSGKDVALSLDDVK

SEQ ID NO: 54 - protein, 14 aa, Artificial Sequence
  Tagged peptide
YSGKEVALSLDDVK

SEQ ID NO: 55 - protein, 14 aa, Artificial Sequence
  Tagged peptide
YSGKFVALSLDDVK

SEQ ID NO: 56 - protein, 14 aa, Artificial Sequence
  Tagged peptide
YSGKGVALSLDDVK

SEQ ID NO: 57 - protein, 14 aa, Artificial Sequence
  Tagged peptide
YSGKHVALSLDDVK

SEQ ID NO: 58 - protein, 14 aa, Artificial Sequence
  Tagged peptide
YSGKIVALSLDDVK

SEQ ID NO: 59 - protein, 14 aa, Artificial Sequence
  Tagged peptide
YSGKKVALSLDDVK

SEQ ID NO: 60 - protein, 14 aa, Artificial Sequence
  Tagged peptide
YSGKLVALSLDDVK

SEQ ID NO: 61 - protein, 14 aa, Artificial Sequence
  Tagged peptide
YSGKMVALSLDDVK

SEQ ID NO: 62 - protein, 14 aa, Artificial Sequence
  Tagged peptide
YSGKNVALSLDDVK

SEQ ID NO: 63 - protein, 14 aa, Artificial Sequence
  Tagged peptide
YSGKPVALSLDDVK

SEQ ID NO: 64 - protein, 14 aa, Artificial Sequence
  Tagged peptide
YSGKQVALSLDDVK

SEQ ID NO: 65 - protein, 14 aa, Artificial Sequence
  Tagged peptide
YSGKRVALSLDDVK

SEQ ID NO: 66 - protein, 14 aa, Artificial Sequence
  Tagged peptide
YSGKSVALSLDDVK

SEQ ID NO: 67 - protein, 14 aa, Artificial Sequence
  Tagged peptide
YSGKTVALSLDDVK

SEQ ID NO: 68 - protein, 14 aa, Artificial Sequence
  Tagged peptide
YSGKVVALSLDDVK

SEQ ID NO: 69 - protein, 14 aa, Artificial Sequence
  Tagged peptide
YSGKWVALSLDDVK

SEQ ID NO: 70 - protein, 14 aa, Artificial Sequence
  Tagged peptide
YSGKYVALSLDDVK

SEQ ID NO: 71 - protein, 16 aa, Artificial Sequence
  Tagged peptide
FYGMALDGYAPKSAYR

SEQ ID NO: 72 - protein, 18 aa, Artificial Sequence
  Tagged peptide
YLLHASIERPSPDKSAYR

SEQ ID NO: 73 - protein, 14 aa, Artificial Sequence
  Synthetic peptide
YLLHASIERPSPDK

SEQ ID NO: 74 - protein, 4 aa, Artificial Sequence
  Peptide-tag
SAAG

(19) United States
(12) Patent Application Publication
(10) Pub. No.: US 2016/0223562 A1
(43) Pub. Date: Aug. 4, 2016

(54) Method for Determining the Concentration of a Peptide

(71) Applicant: JPT Peptide Technologies GmbH, Berlin (DE)

(72) Inventors: Johannes Zerweck, Berlin (DE); Mike Schutkowski, Querfurt (DE); Holger Wenschuh, Berlin (DE)

(21) Appl. No.: 15/074,214

(22) Filed: Mar. 18, 2016

Related U.S. Application Data
(62) Division of application No. 13/634,197, filed on Nov. 8, 2012, now abandoned.

(30) Foreign Application Priority Data
Mar. 12, 2010 (EP) 10002664.0

(51) Int. Cl. G01N 33/68

(52) U.S. Cl. CPC G01N 33/6848; G01N 2458/15; G01N 2560/00

(57) ABSTRACT

The present invention is related to a method for preparing an internal peptide standard, comprising a) providing a first tag, and b) coupling of a first peptide to the first tag, whereby the first peptide comprises an amino acid sequence, or a) providing a first tag and b) coupling to the first tag the amino acids forming the first peptide comprising an amino acid sequence, whereby the C-terminal end of the amino acid sequence of the first peptide corresponds to the C-terminal end generated by a sequence-specific hydrolysis of an amide bond, ester bond or thioester bond preferably of an amide bond of a peptide.

### RELATED APPLICATIONS

This application is a divisional of U.S. patent application Ser. No. 13/634,197, filed Nov. 8, 2012; which is the U.S. national phase of International Patent Application No. PCT/EP2011/001167, filed Mar. 9, 2011; which claims the benefit of priority to European Patent Application serial number 10002664.0, filed Mar. 12, 2010.

The present invention is related to a method for determining the presence and/or quantity of a target polypeptide of which a first peptide is a proteotypic peptide, in at least one mixture of different polypeptides, a method for preparing an internal peptide standard, a method for preparing a library of internal peptide standards, wherein the library consists of a plurality of species of an internal peptide standard, an internal peptide standard obtainable by a method for preparing an internal peptide standard, and a panel of internal standards obtainable by a method for preparing an internal peptide standard.

There is a need in the art to provide novel methods for the quantification of proteins and modified proteins from cell lysates. Furthermore, there is a need to provide novel methods for the accurate comparison of protein expression levels between cells in two different states, particularly for comparison of low abundance proteins.

The current standard for protein detection and more specifically protein quantification is based on immunoreactive detection (Western analysis). However, this technique requires the availability of an appropriately specific antibody. In addition, many antibodies only recognize proteins in an unfolded (denatured) form, cross-reactivity can be severely limiting, and quantification is generally relative.

The development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997). However, while these approaches dramatically accelerate protein identification, quantities of the analyzed proteins cannot be easily determined, and these methods have not been shown to substantially alleviate the dynamic range problem also encountered by the 2DE/MS/MS approach. Therefore, low abundance proteins in complex samples are also difficult to analyze by the microcapillary LC/MS/MS method without their prior enrichment.

Another methodology has recently been described. ICAT reagent technology makes use of a class of chemical reagents called isotope coded affinity tags (ICAT). These reagents exist in isotopically heavy and light forms which are chemically identical with the exception of eight deuterium or hydrogen atoms, respectively. Proteins from two cells lysates can be labeled independently with one or the other ICAT reagent at cysteinyl residues. After mixing and proteolysing the lysates, the ICAT-labeled peptides are isolated by affinity to a biotin molecule incorporated into each ICAT reagent. ICAT-labeled peptides are analyzed by LC-MS/MS where they elute as heavy and light pairs of peptides. Quantification is performed by determining the relative expression ratio relating to the amount of each ICAT-labeled peptide pair in the sample.

Identification of each ICAT-labeled peptide is performed by a second stage of mass spectrometry (MS/MS) and sequence database searching. The end result is relative protein expression ratios on a large scale. The major drawback to this technique are 1) quantification is only relative; 2) specialized chemistry is required, and 3) database searches are hindered by the presence of the large ICAT reagent molecule, and 4) relative amounts of post-translationally modified (e. g., phosphorylated) proteins are transparent to analysis.

International patent application WO 03/016861 discloses a method for absolute quantification of proteins directly from cell lysates whereby an internal peptide standard is used. The method requires the addition of a known amount of the internal standard peptide.

The problem underlying the instant invention is to provide a method for determining the presence of a target polypeptide in a mixture of different polypeptides. A further problem underlying the instant invention is to provide a method for determining the quantity of a target polypeptide in a mixture of different polypeptides.

A further problem underlying the instant invention is to provide a method for the generation of an internal peptide standard, whereby the internal peptide standard is particularly suitable for use in a method for determining the presence and/or quantity of a target polypeptide in a mixture of different polypeptides.

A still further problem underlying the instant invention is to provide an internal peptide standard and a panel of internal standard peptides, respectively, which is particularly suitable for use in a method for determining the presence and/or quantity of a target polypeptide in a mixture of different polypeptides.

These and other problems underlying the present invention are solved by the subject matter of the attached independent claims. Preferred embodiments may be taken from the attached dependent claims and the following description.

In a first aspect, which is also the first embodiment of the first aspect, the problem underlying the invention is solved by a method for preparing an internal peptide standard, comprising
a) providing a first tag, and
b) coupling of a first peptide to the first tag, whereby the first peptide comprises an amino acid sequence,
or
a) providing a first tag and
b) coupling to the first tag the amino acids forming the first peptide comprising an amino acid sequence,
   whereby the C-terminal end of the amino acid sequence of the first peptide corresponds to the C-terminal end generated by a sequence-specific hydrolysis of an amide bond, ester bond or thioester bond preferably of an amide bond of a peptide.

In a second embodiment of the first aspect which is also an embodiment of the first embodiment of the first aspect, the sequence-specific hydrolysis occurs by means of a reaction selected from the group comprising a reaction of a proteolytic enzyme, a sequence-specific chemical reaction or a sequence-specific physical treatment.

In a third embodiment of the first aspect which is also an embodiment of the second embodiment of the first aspect, the proteolytic enzyme is selected from the group comprising hydrolases, peptidases, proteases, trypsin, thrombin, V8 protease, prolyl endopeptidase, subtilisin and chymotrypsin and eslastase.

In a fourth embodiment of the first aspect which is also an embodiment of the second embodiment of the first aspect, the sequence-specific chemical reaction is selected from the group comprising treatment with cyanogens halides like cyanogen bromide yielding C-terminal homoserine lactones and selective acidic hydrolysis of aspartyl-prolyl bonds yielding C-terminal aspartic acid residues.

In a fifth embodiment of the first aspect which is also an embodiment of the first to the fourth embodiment of the first aspect, the C-terminal end is an amino acid selected from the group comprising arginine, lysine, glutamic acid and proline.

In a sixth embodiment of the first aspect which is also an embodiment of the first to the fifth embodiment of the first aspect, the internal peptide standard is for use in a method for determining the presence and/or quantity of a target polypeptide in at least one mixture of different polypeptides and/or quantity of a purified polypeptide.

In a seventh embodiment of the first aspect which is also an embodiment of the first to the sixth embodiment of the first aspect, the first tag is selected from the group comprising a peptide, a protease substrate and a leaving group.

In an eight embodiment of the first aspect which is also an embodiment of the first to the seventh embodiment of the first aspect, the first tag is a peptide consisting of 2 to 10 amino acid residues, preferably 2 to 6 amino acid residues, more preferably 2 to 4 amino acid residues and most preferably 3 amino acid residues.

In a ninth embodiment of the first aspect which is also an embodiment of the eighth embodiment of the first aspect, the first tag comprises at least one biogenic amino acid residue.

In a tenth embodiment of the first aspect which is also an embodiment of the eighth embodiment of the first aspect, the first tag comprises at least one non-biogenic amino acid residue.

In an eleventh embodiment of the first aspect which is also an embodiment of the eighth embodiment of the first aspect, the first tag comprises at least one D-amino acid residue.

In a twelfth embodiment of the first aspect which is also an embodiment of the eighth to the eleventh embodiment of the first aspect, at least one of the amino acid residues of the first tag is selected from the group comprising alpha-amino acid residues, beta-amino acid residues, gamma-amino acid residues and delta-amino acid residues.

In a 13th embodiment of the first aspect which is also an embodiment of the seventh to the twelfth embodiment of the first aspect, the first tag is a peptide and wherein at least one of the amino acid residues of the peptide forming the first tag comprises an isotope composition which is different from the isotope composition obtained in a non-isotope discriminating method of synthesis of the peptide.

In a 14th embodiment of the first aspect which is also an embodiment of the seventh embodiment of the first aspect, the first tag is a part of a protease substrate, whereby the protease substrate is preferably selected from the group comprising peptides, aromatic amides, aromatic esters or thioesters, aliphatic amides, esters or thioesters.

In a 15th embodiment of the first aspect which is also an embodiment of the seventh embodiment of the first aspect, the first tag is a leaving group, whereby the leaving group is preferably selected from the group comprising peptides, aromatic amines, phenols or thiophenols, aliphatic amines, aliphatic alcohols or aliphatic thioles.

In a 16th embodiment of the first aspect which is also an embodiment of the first to the 15th embodiment of the first aspect, the first tag comprises a reactive group which allows the specific coupling of a label.

In a 17th embodiment of the first aspect which is also an embodiment of the 16th embodiment of the first aspect, the label is coupled to the first tag after the coupling of the first tag to the first peptide.

In an 18th embodiment of the first aspect which is also an embodiment of the first to the 17th embodiment of the first aspect, the first tag comprises a label.

In a 19th embodiment of the first aspect which is also an embodiment of the 16th to the 18th embodiment of the first aspect, the label is selected from the group comprising a mass label, a fluorescence label, and a UV label.

In a 20th embodiment of the first aspect which is also an embodiment of the first to the 19th embodiment of the first aspect, the label is a mass label contained in a cage compound.

In a 21st embodiment of the first aspect which is also an embodiment of the 19th to the 20th embodiment of the first aspect, the mass label is an isotope label, wherein the isotope is preferably selected from the group comprising C isotopes, N isotopes, Cl isotopes and Br isotopes.

In a 22nd embodiment of the first aspect which is also an embodiment of the 19th embodiment of the first aspect, the fluorescent label is selected from the group comprising β-7-methoxy-coumaryl-alanine, coumaryl derivatives, naphthylamine derivatives, hydroxyl and thiol derivatives of naphthalene, amino-, hydroxyl- or thiol-fluoresceine derivatives, amino-, hydroxyl- or thiol-rhodamine derivatives, and aminobenzoic acid derivatives.

In a 23rd embodiment of the first aspect which is also an embodiment of the 19th embodiment of the first aspect, the UV label is selected from the group comprising substituted aromatic amines, amides, phenols, thiophenols, esters, thioesters like substituted anilines, substituted anilides, substituted phenols, substituted thiophenols, substituted naphthylamines, substituted naphthylamides, substituted thioarylesters, substituted arylesters and substituted aliphatic amines, amides, alcohols, thiols, esters, thioesters like substituted primary and secondary amines, substituted secondary and tertiary amides, substituted alcohols, substituted thiols, substituted thioesters, substituted esters.

In a 24th embodiment of the first aspect which is also an embodiment of the first to the 23rd embodiment of the first aspect, the first tag or a first part of the first tag is removable after the coupling to the first peptide.

In a 25th embodiment of the first aspect which is also an embodiment of the 24th embodiment of the first aspect, the first tag or a first part thereof is removable by a means selected from the group comprising chemical cleavage, enzymatic cleavage and physical cleavage.

In a 26th embodiment of the first aspect which is also an embodiment of the 24th to the 25th embodiment of the first aspect, the removal of the first tag or the first part thereof is sequence-specific.

In a 27th embodiment of the first aspect which is also an embodiment of the first to the 26th embodiment of the first aspect, the first tag or the first part thereof provides a signal in a detection method.

In a 28th embodiment of the first aspect which is also an embodiment of the first to the 27th embodiment of the first aspect, the signal is provided by the label.

In a 29th embodiment of the first aspect which is also an embodiment of the 28th embodiment of the first aspect, the signal is a specific signal which is specifically indicative of the first tag or the first part thereof.

In a 30th embodiment of the first aspect which is also an embodiment of the 27th to the 29th embodiment of the first aspect, the signal is a quantitative signal.

In a 31st embodiment of the first aspect which is also an embodiment of the 27th to the 30th embodiment of the first aspect, the signal is proportionate to the first tag or the first part thereof, preferable in accordance with a 1:1 stoichiometry.

In a 32nd embodiment of the first aspect which is also an embodiment of the 27th to the 31st embodiment of the first aspect, the detection method is selected from the group comprising fluorescence detection, UV detection and mass detection.

In a 33rd embodiment of the first aspect which is also an embodiment of the first to the 32nd embodiment of the first aspect, the amino acid sequence of the first peptide corresponds to an amino acid sequence of a proteotypic polypeptide or a fragment of the proteotypic polypeptide.

In a 34th embodiment of the first aspect which is also an embodiment of the first to the 32nd embodiment of the first aspect, the amino acid sequence of the first peptide corresponds to the amino acid sequence of a plurality of proteotypic polypeptides.

In a 35th embodiment of the first aspect which is also an embodiment of the first to the 34th embodiment of the first aspect, the coupling of the first peptide to the first tag is performed by synthesizing the amino acid sequence of the first peptide to the first tag.

In a 36th embodiment of the first aspect which is also an embodiment of the 35th embodiment of the first aspect, the amino acids of the amino acid sequence of the first peptide are sequentially added to the first tag.

In a 37th embodiment of the first aspect which is also an embodiment of the first to the 36th embodiment of the first aspect, the first tag is immobilized to a surface.

In a 38th embodiment of the first aspect which is also an embodiment of the first to the 36th embodiment of the first aspect, the first tag is synthesized at a surface prior to the coupling of the first peptide to the first tag.

In a 39th embodiment of the first aspect which is also an embodiment of the first to the 38th embodiment of the first aspect, the first tag is coupled to the C-terminal end of the first peptide.

In a 40th embodiment of the first aspect which is also an embodiment of the first to the 39th embodiment of the first aspect, the first peptide comprises a second tag.

In a 41st embodiment of the first aspect which is also an embodiment of the first to the 40th embodiment of the first aspect, the second tag is coupled to the N-terminal end of the first peptide, preferably to an N-terminal amino acid of the first peptide.

In a 42nd embodiment of the first aspect which is also an embodiment of the 41st embodiment of the first aspect, the second tag is coupled to the N-terminal amino acid of the first peptide upon completion of the synthesis or coupling of the full-length amino acid sequence of the first peptide.

In a 43rd embodiment of the first aspect which is also an embodiment of the 41st embodiment of the first aspect, the second tag is coupled to the N-terminal amino acid of the amino acid sequence of the first peptide and said second tag is coupled, preferably subsequently to the first tag, to the first peptide.

In a 44th embodiment of the first aspect which is also an embodiment of the 40th to the 43rd embodiment of the first aspect, the second tag is different from the first tag.

In a 45th embodiment of the first aspect which is also an embodiment of the 40th to the 44th embodiment of the first aspect, the second tag is selected from the group comprising a peptide, a protease substrate and a leaving group.

In a 46th embodiment of the first aspect which is also an embodiment of 45th embodiment of the first aspect, the second tag is a peptide consisting of 2 to 10 amino acid residues, preferably 2 to 6 amino acid residues, more preferably 2 to 4 amino acid residues and most preferably 3 amino acid residues.

In a 47th embodiment of the first aspect which is also an embodiment of the 46th embodiment of the first aspect, the second tag comprises at least one biogenic amino acid residue.

In a 48th embodiment of the first aspect which is also an embodiment of the 46th embodiment of the first aspect, the second tag comprises at least one non-biogenic amino acid residue.

In a 49th embodiment of the first aspect which is also an embodiment of the 46th embodiment of the first aspect, the second tag comprises at least one D-amino acid residue.

In a 50th embodiment of the first aspect which is also an embodiment of the 46th to the 49th embodiment of the first aspect, at least one of the amino acid residues of the second tag is selected from the group comprising alpha-amino acid residues, beta-amino acid residues, gamma-amino acid residues and delta-amino acid residues.

In a 51st embodiment of the first aspect which is also an embodiment of the 45th to the 50th embodiment of the first aspect, the second tag is a peptide and wherein at least one amino acid residues of the peptide forming the second tag comprises an isotope composition which is different from the isotope composition obtained in a non-isotope discriminating method of synthesis of the peptide.

In a 52nd embodiment of the first aspect which is also an embodiment of the 45th embodiment of the first aspect, the second tag is a protease substrate, whereby the protease substrate is preferably selected from the group comprising peptides and peptide derivatives mimicking the N-terminal part of endoprotease substrates.

In a 53rd embodiment of the first aspect which is also an embodiment of the 45th embodiment of the first aspect, the second tag is a leaving group, whereby the leaving group is preferably selected from the group comprising peptides and substituted peptides.

In a 54th embodiment of the first aspect which is also an embodiment of the 40th to the 53rd embodiment of the first aspect, the second tag comprises a reactive group which allows the specific coupling of a label.

In a 55th embodiment of the first aspect which is also an embodiment of the 40th embodiment of the first aspect, the label is coupled to the second tag after the coupling of the first tag to the first peptide.

In a 56th embodiment of the first aspect which is also an embodiment of the 40th to the 55th embodiment of the first aspect, the second tag comprises a label.

In a 57th embodiment of the first aspect which is also an embodiment of the 54th to the 56th embodiment of the first aspect, the label is selected from the group comprising a mass label, a fluorescence label, and a UV label.

In a 58th embodiment of the first aspect which is also an embodiment of the 57th embodiment of the first aspect, the label is a mass label contained in a cage compound.

In a 59th embodiment of the first aspect which is also an embodiment of the 57th to the 58th embodiment of the first aspect, the mass label is an isotope label, wherein the isotope is preferably selected from the group comprising C isotopes, N isotopes, Cl isotopes and Br isotopes.

In a 60th embodiment of the first aspect which is also an embodiment of the 57th embodiment of the first aspect, the fluorescent label is selected from the group comprising β-7-methoxy-coumaryl-alanine, coumaryl derivatives, naphthylamine derivatives, hydroxyl and thiol derivatives of naphthalene, amino-, hydroxyl- or thiol-fluoresceine derivatives, amino-, hydroxyl- or thiol-rhodamine derivatives, and aminobenzoic acid derivatives.

In a 61st embodiment of the first aspect which is also an embodiment of the 57th embodiment of the first aspect, the UV label is selected from the group comprising substituted aromatic amines, amides, phenols, thiophenols, esters, thioesters like substituted anilines, substituted anilides, substituted phenols, substituted thiophenols, substituted naphthylamines, substituted naphthylamides, substituted thioarylesters, substituted arylesters and substituted aliphatic amines, amides, alcohols, thiols, esters, thioesters like substituted primary and secondary amines, substituted secondary and tertiary amides, substituted alcohols, substituted thiols, substituted thioesters, substituted esters.

In a 62nd embodiment of the first aspect which is also an embodiment of the 40th to the 61st embodiment of the first aspect, the second tag or a first part of the second tag is removable after the coupling to the first peptide.

In a 63rd embodiment of the first aspect which is also an embodiment of the 62nd embodiment of the first aspect, the second tag or the first part thereof is removable by a means selected from the group comprising chemical cleavage, enzymatic cleavage and physical cleavage.

In a 64th embodiment of the first aspect which is also an embodiment of the 62nd to the 63rd embodiment of the first aspect, the removal of the second tag or the first part thereof is sequence-specific.

In a 65th embodiment of the first aspect which is also an embodiment of the 40th to the 64th embodiment of the first aspect, the second tag or the first part thereof provides a signal in a detection method.

In a 66th embodiment of the first aspect which is also an embodiment of the 65th embodiment of the first aspect, the signal is provided by the label.

In a 67th embodiment of the first aspect which is also an embodiment of the 66th embodiment of the first aspect, the signal is a specific signal which is specifically indicative of the second tag or the first part thereof.

In a 68th embodiment of the first aspect which is also an embodiment of the 65th to the 67th embodiment of the first aspect, the signal is a quantitative signal.

In a 69th embodiment of the first aspect which is also an embodiment of the 65th to the 68th embodiment of the first aspect, the signal is proportionate to the first tag or the first part thereof.

In a 70th embodiment of the first aspect which is also an embodiment of the 65th to the 69th embodiment of the first aspect, the detection method is selected from the group comprising fluorescence detection, UV detection and mass detection.

In a 71st embodiment of the first aspect which is also an embodiment of the 40th to the 70th embodiment of the first aspect, the first tag and the second tag comprise a different label.

In a 72nd embodiment of the first aspect which is also an embodiment of the 40th to the 71st embodiment of the first aspect, the second tag comprises an anchor moiety.

In a 73rd embodiment of the first aspect which is also an embodiment of the 72nd embodiment of the first aspect, the anchor moiety is suitable for attaching the second tag and/or the internal peptide standard to a surface.

In a 74th embodiment of the first aspect which is also an embodiment of the 72nd to the 73rd embodiment of the first aspect, the anchor moiety allows the reversible attachment of the second tag and/or the internal peptide standard to a surface.

In a 75th embodiment of the first aspect which is also an embodiment of the 72nd to the 74th embodiment of the first aspect, the anchor moiety is selected from the group comprising biotin, desthiobiotin, avidin and streptavidin, or is a chemical group allowing chemoselective reaction with the appropriate, i.e. corresponding reactive function on the surface.

In a 76th embodiment of the first aspect which is also an embodiment of the 72nd embodiment of the first aspect, the anchor moiety allows the irreversible attachment of the second tag and/or the internal peptide standard to a surface, whereby preferably the irreversible attachment is a covalent attachment.

In a 77th embodiment of the first aspect which is also an embodiment of the 72nd to the 76th embodiment of the first aspect, the anchor moiety is attached to a linker, whereby the linker is arranged between the anchor moiety and the second tag.

In a 78th embodiment of the first aspect which is also an embodiment of the 77th embodiment of the first aspect, the linker is selected from the group comprising N-(3-{2-[2-(3-amino-propoxy)-ethoxy]-ethoxy}-propyl)-succinamic acid or alkanes either branched or linear chains, preferably those composed of 2-30 carbon atoms, more preferably those composed of 4-20 carbon atoms and most preferably those composed of 4-10 carbon atoms; or polyethers, preferably polymers composed of 1-10, more preferably 2-5 ethylenoxide or polypropylene oxide units, or polyalcohols, branched or unbranched, or polyurethanes, polyhydroxy acids, polycarbonates, polyimides, polyamides, polyester, polysulfones, preferably those composed of 1-100 monomeric units, more preferably those composed of 1-50 monomeric units and most preferably those composed of 1-20 monomeric units, or combinations of the said alkanes with the said polyurethanes, polyhydroxy acids, polycarbonates, polyimides, polyamides, polyester, polysulfones, or diaminoalkanes either branched or linear chains, preferably those composed of 2-30 carbon atoms, more preferably those composed of 2-20 carbon atoms and most preferably those composed of 2-10 carbon atoms, as well as combinations of diaminoalkanes with polyethers, preferably those composed of 2-30 carbon atoms, more preferably those composed of 2-20 carbon atoms and most preferably those composed of 2-10 carbon atoms like succinic acid or glutaric acid, amino acids and peptides preferably composed of 1-20 residues or more preferably 1-10 residues or most preferably 1-3 residues, preferably the linker is a combination of a dicarboxylic acid with a diamino polyether like N-(3-{2-[2-(3-amino-propoxy)-ethoxy]-ethoxy}-propyl)-succinamic acid.

In a 79th embodiment of the first aspect which is also an embodiment of the 72nd to the 78th embodiment of the first aspect, the second tag comprises a peptide comprising an amino acid sequence, whereby the linker is arranged between the anchor moiety and the N-terminal end, preferably between the N-terminal amino acid residue of the second tag.

In a 80th embodiment of the first aspect which is also an embodiment of the first to the 79th embodiment of the first aspect, the internal peptide standard is purified.

In a 81st embodiment of the first aspect which is also an embodiment of the 40th to the 80th embodiment of the first aspect, the internal peptide standard comprises a second tag, a first peptide and a first tag, wherein the internal peptide standard is contained in a mixture of peptides, wherein the internal peptide standard is immobilized to a surface, and wherein the internal peptide standard specifically interacts with the surface through the anchor moiety of the internal peptide standard.

In a 82nd embodiment of the first aspect which is also an embodiment of the 80th to the 81st embodiment of the first aspect, the peptides different from the internal peptide standard are removed from the mixture.

In a 83rd embodiment of the first aspect which is also an embodiment of the 81st to the 82nd embodiment of the first aspect, the internal peptide standard is removed from the surface and transferred to a reaction vessel.

In a 84th embodiment of the first aspect which is also an embodiment of the first to the 83rd embodiment of the first aspect, preferably an embodiment of the first to the 67th embodiment of the first aspect, the first and/or the second tag is/are removed from the first peptide.

In a 85th embodiment of the first aspect which is also an embodiment of the first to the 84th embodiment of the first aspect, the amount of the first and/or the second tag is/are quantified.

In a second aspect, which is also the first embodiment of the second aspect, the problem underlying the invention is solved by a method for preparing a library of internal peptide standards, wherein the library consists of a plurality of species of an internal peptide standard comprising
a) individually preparing a plurality of species of an internal peptide standard, whereby each species of an internal peptide standard is prepared according to any of the embodiments of the first aspect; and
b) optionally mixing the individually prepared species of an internal peptide standard,
   wherein the species of an internal peptide standard differ from each other in the first and/or second tag and/or the amino acid sequence of first peptide.

In a second embodiment of the second aspect which is also an embodiment of first embodiment of the second aspect, the first and/or the second tag comprises a peptide having an amino acid sequence and the amino acid sequence of each species of the library of internal peptide standards is different from the amino acid sequence of the other species of the library of internal peptide standards.

In a third embodiment of the second aspect which is also an embodiment of first and the second embodiment of the second aspect, each species of an internal peptide standard of the library of internal peptide standards comprises a proteotypic peptide.

In a third aspect, which is also the first embodiment of the third aspect, the problem underlying the invention is solved by an internal peptide standard obtainable by a method according to any embodiment of the first aspect.

In a fourth aspect, which is also the first embodiment of the fourth aspect, the problem underlying the invention is solved by a panel of internal peptide standards obtainable by a method according to any embodiment of the second aspect.

In a fifth aspect, which is also the first embodiment of the fifth aspect, the problem underlying the invention is solved by a method for determining the presence and/or quantity of a target polypeptide of which a first peptide is a proteotypic peptide, in at least one mixture of different polypeptides, comprising:
a) providing a mixture of different polypeptides preferably containing the target polypeptide;
b) adding a quantity of an internal peptide standard according to any embodiment of the third aspect or of a panel of internal peptide standards according to any embodiment of the fourth aspect, thereby generating a spiked mixture;
c) treating the spiked mixture with a means for sequence-specific hydrolysis to generate a plurality of peptides from the mixture of different polypeptides and to cleave off the first tag and/or the second tag from the internal peptide standard/standards thus releasing the first peptide, whereby the plurality of peptides comprises the proteotypic peptide derived from the different polypeptides and the first peptide released from the internal standard;
d) determining the amount of the first and/or second tag contained in the spiked mixture and therefrom the quantity of the internal standard/standards added in step b);
e) determining the ratio of the first peptide to the proteotypic peptide derived from the different polypeptides, each generated in step c); and
f) calculating from the ratio and the quantity of the internal standard/standards determined in step d) the quantity of the target polypeptide in the mixture of different polypeptides,
   wherein either the proteotypic peptide derived from the different polypeptides or the first peptide of the internal peptide standard/standards comprise a mass label.

In a second embodiment of the fifth aspect which is also an embodiment of first embodiment of the fifth aspect, the mass label is selected from the group comprising C isotopes and N isotopes.

In a third embodiment of the fifth aspect which is also an embodiment of first and the second embodiment of the fifth aspect, the ratio of the first peptide released from the internal peptide standard in step c) of claim 91 to the proteotypic peptide derived from the different polypeptides in step c) of claim 91 is determined by mass spectrometry.

In a fourth embodiment of the fifth aspect which is also an embodiment of first to the third embodiment of the fifth aspect, the mass spectrometry is multistage mass spectrometry.

In a fifth embodiment of the fifth aspect which is also an embodiment of first to the fourth embodiment of the fifth aspect, a peptide signature obtained by fragmentation and subsequent analysis in a tandem mass spectrometer which is diagnostic for the presence of the peptide is known for the proteotypic peptide.

In a sixth embodiment of the fifth aspect which is also an embodiment of first to the fifth embodiment of the fifth aspect, the means for sequence-specific hydrolysis is selected from a proteolytic enzyme, a sequence-specific chemical reaction or a sequence-specific physical treatment.

In a seventh embodiment of the fifth aspect which is also an embodiment of first to the sixth embodiment of the fifth aspect, the internal peptide standard according to any embodiment of the third aspect or the panel of internal peptide standards according to any embodiment of the fourth aspect are not purified prior to adding to the mixture of different polypeptides preferably containing the target polypeptide.

In an eighth embodiment of the fifth aspect which is also an embodiment of first to the sixth embodiment of the fifth aspect, the internal peptide standard according to any embodiment of the third aspect or the panel of internal peptide standards according to any embodiment of the fourth aspect are purified prior to adding to the mixture of different polypeptides preferably containing the target polypeptide.

In a ninth embodiment of the fifth aspect which is also an embodiment of eighth embodiment of the fifth aspect, the purification is based on a selective removal of the internal peptide standard/standards from by-products of the synthesis of said internal peptide standard/standards by means of the second tag.

In a tenth embodiment of the fifth aspect which is also an embodiment of first to the ninth embodiment of the fifth aspect, the tag the amount of which is determined in step d) comprises a fluorescence label and the amount of the tag is determined by measuring the fluorescence signal of the label.

In an eleventh embodiment of the fifth aspect which is also an embodiment of first to the ninth embodiment of the fifth aspect, the tag the amount of which is determined in step d) comprises a UV label and the amount of the tag is determined by measuring the UV signal of the label.

In a twelfth embodiment of the fifth aspect which is also an embodiment of first to the eleventh embodiment of the fifth aspect, after step c) and prior to step d) the treated spiked mixture is subjected to a separation step where the first and the second tag is/are separated from components of the treated spiked mixture.

In a 13th embodiment of the fifth aspect which is also an embodiment of first to the eleventh embodiment of the fifth aspect, after step c) and prior to step e) the treated spiked mixture is subjected to a separation step where the first peptide and the proteotypic peptide derived from the different polypeptides are separated from components of the treated spiked mixture.

In a sixth aspect, which is also the first embodiment of the sixth aspect, the problem underlying the invention is solved by a method for determining the presence and/or quantity of a target polypeptide of which a first peptide is a proteotypic peptide, in at least one mixture of different polypeptides, comprising:

a) providing a mixture of different polypeptides preferably containing the target polypeptide;
b) providing an internal peptide standard according to any embodiment of the third aspect or a panel of internal peptide standards according to any embodiment of the fourth aspect, treating the internal peptide standard or the panel of internal peptide standard with a means for sequence-specific hydrolysis to cleave off the first tag and/or the second tag from the internal peptide standard or the panel of internal peptide standards thus releasing the first peptide;
either
ca1) adding the reaction mixture obtained in the performance of step b) to the mixture of different polypeptides of step a) and
ca2) treating the mixture obtained in the performance of step ca1) with a means for sequence-specific hydrolysis to generate a plurality of peptides from the mixture of different polypeptides, whereby the plurality of peptides comprises the proteotypic peptide;
or
cb1) treating the mixture of different polypeptides of step a) with a means for sequence-specific hydrolysis to generate a plurality of peptides from the mixture of different polypeptides, whereby the plurality of peptides comprises the proteotypic peptide; and
cb2) adding the reaction mixture obtained in the performance of step b) to the mixture obtained in the performance of the step cb1)
whereby after step ca2) and cb2), respectively, a spiked mixture is obtained;
d) determining the amount of the first and/or second tag contained in the spiked mixture and therefrom the quantity of the internal standard/standards added in step b);
e) determining in the spiked mixture the ratio of the first peptide to the proteotypic peptide derived from the different polypeptides; and
f) calculating from the ratio and the quantity of the internal standard/standards determined in step d) the quantity of the target polypeptide in the mixture of different polypeptides,
wherein either the proteotypic peptide derived from the different polypeptides or the first peptide of the internal peptide standard/standards comprise a mass label.

In a second embodiment of the sixth aspect which is also an embodiment of the first embodiment of the sixth aspect, the mass label is selected from the group comprising C isotopes and N isotopes.

In a third embodiment of the sixth aspect which is also an embodiment of the first and the second embodiment of the sixth aspect, the ratio of the first peptide released from the internal peptide standard to the proteotypic peptide derived from the different polypeptides is determined by mass spectrometry.

In a fourth embodiment of the sixth aspect which is also an embodiment of the first to the third embodiment of the sixth aspect, the mass spectrometry is multistage mass spectrometry.

In a fifth embodiment of the sixth aspect which is also an embodiment of the first to the fourth embodiment of the sixth aspect, a peptide signature obtained by fragmentation and subsequent analysis in a tandem mass spectrometer which is diagnostic for the presence of the peptide is known for the proteotypic peptide.

In a sixth embodiment of the sixth aspect which is also an embodiment of the first to the fifth embodiment of the sixth aspect, the means for sequence-specific hydrolysis is selected from a proteolytic enzyme, a sequence-specific chemical reaction or a sequence-specific physical treatment.

In a seventh embodiment of the sixth aspect which is also an embodiment of the first to the sixth embodiment of the sixth aspect, the internal peptide standard according to any embodiment of the third aspect or the panel of internal peptide standards according to any embodiment of the fourth aspect are not purified prior to adding to the mixture of different polypeptides preferably containing the target polypeptide.

In an eighth embodiment of the sixth aspect which is also an embodiment of the first to the sixth embodiment of the sixth aspect, the internal peptide standard according to any embodiment of the third aspect or the panel of internal peptide standards according to any embodiment of the fourth aspect are purified prior to adding to the mixture of different polypeptides preferably containing the target polypeptide.

In a ninth embodiment of the sixth aspect which is also an embodiment of the eighth embodiment of the sixth aspect, the purification is based on a selective removal of the internal peptide standard/standards from by-products of the synthesis of said internal peptide standard/standards by means of the second tag.

In a tenth embodiment of the sixth aspect which is also an embodiment of the first to the ninth embodiment of the sixth aspect, the tag the amount of which is determined in step d) comprises a fluorescence label and the amount of the tag is determined by measuring the fluorescence signal of the label.

In an eleventh embodiment of the sixth aspect which is also an embodiment of the first to the ninth embodiment of the sixth aspect, the tag the amount of which is determined in step d) comprises a UV label and the amount of the tag is determined by measuring the UV signal of the label.

In a twelfth embodiment of the sixth aspect which is also an embodiment of the first to the tenth embodiment of the sixth aspect, prior to step d) the spiked mixture is subjected to a separation step where the first and the second tag is/are separated from components of the treated spiked mixture.

In a 13th embodiment of the sixth aspect which is also an embodiment of the first to the eleventh embodiment of the sixth aspect, prior to step e) the spiked mixture is subjected to a separation step where the first peptide and the proteotypic peptide derived from the different polypeptides are separated from components of the treated spiked mixture.

In a 14th embodiment of the sixth aspect which is also an embodiment of the first to the 13th embodiment of the sixth aspect, the means for sequence-specific hydrolysis used in step b) is removed from the mixture obtained in the performance of step b) prior to performing step ca1) or cb1).

In a seventh aspect, which is also the first embodiment of the seventh aspect, the problem underlying the invention is solved by a method for determining the presence and/or quantity of a target polypeptide of which a first peptide is a proteotypic peptide, in at least one mixture of different polypeptides, comprising:

a) providing an internal peptide standard according to any embodiment of the third aspect or a panel of internal peptide standards according to any embodiment of the fourth aspect, treating the internal peptide standard or the panel of internal peptide standard with a means for sequence-specific hydrolysis to cleave off the first tag and/or the second tag from the internal peptide standard or the panel of internal peptide standards thus releasing the first peptide;
b) optionally removing the means for sequence-specific hydrolysis;
c) determining the amount of the first and/or second tag contained in the mixture obtained from the performance of step a) of b) and therefrom the amount of the internal standard contained in said mixture;
d) providing a mixture of different polypeptides preferably containing the target polypeptide;
either
da1) adding a part of the reaction mixture obtained in the performance of step a) or b) to the mixture of different polypeptides of step d) and
da2) treating the mixture obtained in the performance of step da1) with a means for sequence-specific hydrolysis to generate a plurality of peptides from the mixture of different polypeptides, whereby the plurality of peptides comprises the proteotypic peptide;
or
db1) treating the mixture of different polypeptides of step d) with a means for sequence-specific hydrolysis to generate a plurality of peptides from the mixture of different polypeptides, whereby the plurality of peptides comprises the proteotypic peptide; and
db2) adding a part of the mixture obtained in the performance of step a) or b) to the mixture obtained in the performance of the step db1);
whereby after step da2) and db2), respectively, a spiked mixture is obtained;
e) determining in the spiked mixture the ratio of the first peptide to the proteotypic peptide derived from the different polypeptides; and
f) calculating from the ratio and the quantity of the internal standard/standards the quantity of the target polypeptide in the mixture of different polypeptides,
wherein either the proteotypic peptide derived from the different polypeptides or the first peptide of the internal peptide standard/standards comprise a mass label.

In a second embodiment of the seventh aspect which is also an embodiment of the first embodiment of the seventh aspect, the mass label is selected from the group comprising C isotopes and N isotopes.

In a third embodiment of the seventh aspect which is also an embodiment of the first and the second embodiment of the seventh aspect, the ratio of the first peptide released from the internal peptide standard to the proteotypic peptide derived from the different polypeptides is determined by mass spectrometry.

In a fourth embodiment of the seventh aspect which is also an embodiment of the first to the third embodiment of the seventh aspect, the mass spectrometry is multistage mass spectrometry.

In a fifth embodiment of the seventh aspect which is also an embodiment of first to the fourth embodiment of the seventh aspect, a peptide signature obtained by fragmentation and subsequent analysis in a tandem mass spectrometer which is diagnostic for the presence of the peptide is known for the proteotypic peptide.

In a sixth embodiment of the seventh aspect which is also an embodiment of the first to the fifth embodiment of the seventh aspect, the means for sequence-specific hydrolysis is selected from a proteolytic enzyme, a sequence-specific chemical reaction or a sequence-specific physical treatment.

In a seventh embodiment of the seventh aspect which is also an embodiment of the first to the sixth embodiment of the seventh aspect, the internal peptide standard according to any embodiment of the third aspect or the panel of internal peptide standards according to any embodiment of the fourth aspect are not purified prior to adding to the mixture of different polypeptides preferably containing the target polypeptide.

In an eighth embodiment of the seventh aspect which is also an embodiment of the first to the sixth embodiment of the seventh aspect, the internal peptide standard according to any embodiment of the third aspect or the panel of internal peptide standards according to any embodiment of the fourth aspect are purified prior to adding to the mixture of different polypeptides preferably containing the target polypeptide.

In a ninth embodiment of the seventh aspect which is also an embodiment of the eighth embodiment of the seventh aspect, the purification is based on a selective removal of the internal peptide standard/standards from by-products of the synthesis of said internal peptide standard/standards by means of the second tag.

In a tenth embodiment of the seventh aspect which is also an embodiment of the first to the ninth embodiment of the seventh aspect, the tag the amount of which is determined in step c) comprises a fluorescence label and the amount of the tag is determined by measuring the fluorescence signal of the label.

In an eleventh embodiment of the seventh aspect which is also an embodiment of the first to the ninth embodiment of the seventh aspect, the tag the amount of which is determined in step c) comprises a UV label and the amount of the tag is determined by measuring the UV signal of the label.

In a twelfth embodiment of the seventh aspect which is also an embodiment of the first to the eleventh embodiment of the seventh aspect, wherein, in the method,

a) prior to step da1) the reaction mixture obtained in the performance of step a) or b) is subjected to a separation step where the first and the second tag is/are separated from components of said reaction mixture, or
b) prior to step db2) the reaction mixture obtained in the performance of step db1) is subjected to a separation step where the proteotypic peptide derived from the different polypeptides is separated from components of said reaction mixture.

The present inventors have surprisingly found that it is possible to determine the presence of a polypeptide in a mixture of different polypeptides using a particular form of an internal standard. More specifically, the present inventors have surprisingly found that a labelled internal standard is suitable for such purpose and that such labelled internal standard does not need to be quantified prior to adding it to a mixture of different polypeptides, but that it is sufficient to determine its quantity upon having it added to the mixture of different polypeptides. It is, however, also within the present invention that the labelled or non-labelled internal standard is quantified prior to adding it to a mixture of different polypeptides.

The method for determining the presence and/or quantity of a target polypeptide in at least one mixture of different polypeptides of the present invention is based on the concept of proteotypic peptides.

A proteotypic peptide as preferably used herein is a peptide fragment of a polypeptide of interest which is unique for the polypeptide in a mixture of polypeptides. Because of this, the identification and quantification, respectively, of the proteotypic peptide is directly and unambiguously correlated with the presence and quantity of the polypeptide of interest in the individual mixture of polypeptides. The amino acid sequence of a proteotypic peptide is preferably contained only once in the polypeptide of interest, although also amino acid sequences which are contained in a polypeptide of interest several times, may be suitable to be the amino acid of a proteotypic peptide. In such case, the only prerequisite for the suitability of this kind of amino acid sequence to act and thus be useful as an amino acid sequence of a proteotypic peptide is that there is a defined relationship or stoichiometry between the number of copies of such amino acid sequence contained in the amino acid sequence of the polypeptide of interest.

However, it is also within the present invention that the amino acid sequence of a proteotypic peptide is contained in two or more polypeptides in a mixture of polypeptides. In such case, the proteotypic peptide is correlated with the two or more polypeptides in the mixture of polypeptides. The quantification of the proteotypic peptide in such case is a quantification of the overall amount of the two or more polypeptides contained in the mixture sharing the amino acid sequence of the proteotypic peptide.

Preferably, the length of a proteotypic peptides is from 4 to 40 amino acid residues, preferably 6 to 25 amino acid residues and more preferably 8 to 15 amino acid residues and most preferable 8-12 amino acid residues.

The practicing of the various methods for determining the presence and/or quantity of a target polypeptide of which a first peptide is a proteotypic peptide, in at least one mixture of different polypeptides, preferably requires the use of an internal peptide standard. A first aspect of the instant application is thus a method for preparing an internal peptide standard. In a preferred embodiment the thus generated standard or panel of standards is used in or is for use in a method for determining the presence and/or quantity of a target polypeptide in at least one mixture of different polypeptides and/or quantity of a purified polypeptide, whereby preferably such method is a method for determining the presence and/or quantity of a target polypeptide in at least one mixture of different polypeptides and/or quantity of a purified polypeptide of the invention.

The method for preparing an internal peptide standard according to invention, comprises the following steps
a) providing a first tag, and
b) coupling of a first peptide to the first tag, whereby the first peptide comprises an amino acid sequence.
   whereby the C-terminal end of the amino acid sequence of the first peptide corresponds to the C-terminal end generated by a sequence-specific hydrolysis of an amide bond, ester bond or thioester bond. Preferably the bond is an amide bond and more specifically of an amide bond of a peptide. In this embodiment of the method of the invention, the first peptide is provided as the product of a synthesis, typically in the form of a full length peptide. Methods for the synthesis of peptides are known to the person skilled in the art and, among others, described in Pennington, 1994, Peptide Synthesis Protocols (Methods in Molecular Biology); Benoiton, 2005, Chemistry of Peptide Synthesis.

Alternatively, the method for preparing an internal peptide standard according to invention, comprises the following steps
a) providing a first tag and
b) coupling to the first tag the amino acids forming the first peptide comprising an amino acid sequence,
   whereby the C-terminal end of the amino acid sequence of the first peptide corresponds to the C-terminal end generated by a sequence-specific hydrolysis of an amide bond, ester bond or thioester bond preferably of an amide bond of a peptide. In this embodiment, the individual amino acids forming the first peptide are subsequently attached to the first tag. It is also within the present invention that groups of amino acids forming part of the amino acid sequence of the first peptide are attached to the first tag or to the first tag once one or several of the amino acids forming part of the amino acid sequence of the first peptide have been attached to the first tag.

As used herein, the term C-terminal end of an amino acid sequence preferably means the last, i.e. most C-terminal amino acid residue of an amino acid sequence.

The sequence-specific hydrolysis is performed by means of a reaction selected from the group comprising a reaction of a proteolytic enzyme, a sequence-specific chemical reaction or a sequence-specific physical treatment. This kind of reactions is known to a person skilled in the art, see, e.g., Handbook of Proteolytic Enzymes, Academic Press, 1998; Kaiser and Metzka, 1999.

In the embodiment where the sequence-specific hydrolysis is performed by means of a proteolytic enzyme, the proteolytic enzyme is selected from the group comprising hydrolases especially endoproteinases like trypsin yielding a C-terminal arginine and lysine residue, thrombin mainly yielding a C-terminal arginine residue, V8 protease yielding a C-terminal glutamic acid residue, prolyl endopeptidase yielding a C-terminal proline residue, subtilisin and chymotrypsin mainly yielding a C-terminal phenylalanine, tyrosine, tryptophan, methionine or leucine residue and eslastase yielding a C-terminal alanine, glycine serine or valine residue.

In the embodiment where the sequence-specific hydrolysis is performed by means of a sequence-specific chemical reaction, the sequence specific chemical reaction is preferably selected from the group comprising treatment with cyanogens halides like cyanogen bromide yielding a C-terminal homoserine lactone and selective acidic hydrolysis of aspartyl-prolyl bonds yielding a C-terminal aspartic acid residue.

In the embodiment the sequence-specific hydrolysis is performed by means of a sequence-specific physical treatment, such sequence-specific physical treatment is preferably selected from ionization-caused fragmentation reactions under high vacuum which, for example, may occur in a mass spectrometer (Pfeifer, T., Schierhorn, A., Friedemann, R., Jakob, M., Frank, R., Schutkowski, M., & Fischer G. (1997) Specific Fragmentation of Thioxo Peptides Facilitates the Assignment of the Thioxylated Amino Acid. J. Mass Spec. 32, 1064-1071).

In a preferred embodiment, and preferably irrespective of which kind of means is used for the sequence-specific hydrolysis, the C-terminal end is an amino acid residue. Preferably such amino acid residue is selected from the group comprising arginine, lysine, glutamic acid and proline.

The first peptide comprises an amino acid sequence. In an embodiment, the first peptide consists of an amino acid sequence. To said first peptide a first tag is attached. Preferably such first tag is attached to the first peptide by a covalent bond. In an embodiment, there is a second tag attached to the first peptide. In an embodiment, both a first tag and a second tag are attached to the first peptide. In the embodiment where a first tag is attached to the first peptide and a second tag is attached to the first peptide, preferably the first tag is attached to the C-terminal amino acid residue of the first peptide, and the second tag is attached to the N-terminal amino acid residue of the first peptide.

In the following, various embodiments of the first tag will be described in more detail. If not indicated differently, the various embodiments described for the first tag may also be embodiments of the second tag. If the description of features and embodiments is simply referring to a or the tag, this means that such description of features and embodiments is a description of features and embodiments of both the first tag and the second tag.

In an embodiment the tag is selected from the group comprising a peptide, a protease substrate and a leaving group.

In the embodiment where the tag is a peptide, such peptide consists of 2 to 10 amino acid residues, preferably 2 to 6 amino acid residues, more preferably 2 to 4 amino acid residues and most preferably 3 amino acid residues. As to the chemistry of such amino acids and amino acid residues, respectively, it is to be acknowledged that the individual amino acid may each and independent of the other amino acid residues of the peptide be a biogenic amino acid or a non-biogenic amino acid. Also the individual amino acid may each and independent of the other amino acid residues of the peptide be an L-amino acid or a D-amino acid. It is also within the present invention that Also the individual amino acid may each and independent of the other amino acid residues of the peptide be an alpha-amino acid, a beta-amino acid residue, a gamma-amino acid residue or a delta-amino acid residue. In a embodiment the amino acid sequence of the tag consists of L-amino acid residues. In another embodiment the amino acid sequence of the tag consists of D-amino acid residues. In a embodiment the amino acid sequence of the tag consists of L-alpha amino acid residues. In another embodiment the amino acid sequence of the tag consists of D-alpha amino acid residues.

In an embodiment the tag is a peptide and wherein at least one of the amino acid residues of the peptide forming the tag comprises an isotope composition which is different from the isotope composition obtained in a non-isotope discriminating method of synthesis of the peptide. As preferably used herein, a non-isotope discriminating method of synthesis of the peptide, is a method for the synthesis where the individual isotopes of the atoms and thus the amino acids containing the individual isotopes are not discriminated. As a consequence, the isotopes of the individual atoms are not biased. Such bias can be such that one or several isotopes are enriched. Alternatively, such bias can be such that one or several isotopes is depleted. Finally, the bias can also be such that one species or group of species of isotopes is enriched, whereas another species or group of species of isotopes is depleted. As the isotopes and their distribution play a key role in mass spectrometry used in connection with the method of the invention, the use of a non-isotope discriminating method for the synthesis of the tag is preferred.

In an embodiment, the tag is a protease substrate or part of a protease substrate, whereby the protease substrate is preferably selected from the group comprising peptides, aromatic amides, aromatic esters or thioesters, aliphatic amides, esters or thioesters. In a embodiment the protease substrate is a peptide, whereby preferably the peptide comprises or consists of 1 to 20 amino acid residues, preferably 1 to 10 amino acid residues and more preferably 1 to 5 amino acid residues and most preferable 1-3 amino acid residues.

In an embodiment, the tag comprises a reactive group, preferably a chemically reactive group. Such reactive group allows the coupling of a label to the tag. In an embodiment the coupling occurs by the formation of a chemical bond between the tag and the label. In an alternative embodiment, the label comprises a reactive group, preferably a chemically reactive group. Such reactive group allows the coupling of the tag to the label. In an embodiment the coupling occurs by the formation of a chemical bond between the tag and the label.

As to the coupling of the label to the tag it is within the present invention that the label is coupled to the tag after the coupling of the tag to the first peptide. In an alternative embodiment, the label is coupled to the tag prior to the coupling of the tag to the first peptide.

In an embodiment, of either or both of the first tag and the second tag, the label is part of an amino acid. In accordance therewith, the amino acid is preferably a modified amino acid. Such amino acid, preferably the modified amino acid, is used in the synthesis of the tag. In a preferred embodiment, the tag is a peptide comprising such amino acid. In an embodiment such modified amino acid is meta-Nitro-tyrosin as, e.g., described in Cawley et al., J. Biol. Chem. 278, (2003).

The label attached to the tag, either or both the first tag and the second tag, is preferably selected from the group comprising a mass label, a fluorescence label, and a UV label.

In an embodiment the mass label is contained in a cage compound. Caged compounds are known in the art and, e.g. described in Capello et al., Cancer Biother. Radiopharm. 18, 2003, Storch et al., J. Nucl. Med. 46, 2005.

In the embodiment where the label is a mass label, the mass label is an isotope label, wherein the isotope is preferably selected from the group comprising C isotopes, N isotopes, Cl isotopes and Br isotopes. Preferred C isotopes as ¹³C. Preferred N isotopes are ¹⁵N isotopes. Preferred Br isotopes are ⁷⁹Br and ⁸¹Br. Preferred Cl isotopes are ³⁵Cl and ³⁷Cl.

In the embodiment where the label is a fluorescent label, the fluorescent label is preferably selected from the group comprising β-7-methoxy-coumaryl-alanine, coumaryl derivatives, naphthylamine derivatives, hydroxyl and thiol derivatives of naphthalene, amino-, hydroxyl- or thiol-fluoresceine derivatives, amino-, hydroxyl- or thiol-rhodamine derivatives, and aminobenzoic acid derivatives as described, among others, in Handbook of Proteolytic Enzymes, Academic Press, 1998, or in The Handbook—A Guide to Fluorescent Probes and Labeling Technologies, Invitrogen, invitrogen.com/site/us/en/home/References/Molecular-Probes-The-Handbook.html.

In the embodiment where the label is a UV label, the UV label is preferably selected from the group comprising substituted aromatic amines, amides, phenols, thiophenols, esters, thioesters like substituted anilines, substituted anilides, substituted phenols, substituted thiophenols, substituted naphthylamines, substituted naphthylamides, substituted thioarylesters, substituted arylesters and substituted aliphatic amines, amides, alcohols, thiols, esters, thioesters like substituted primary and secondary amines, substituted secondary and tertiary amides, substituted alcohols, substituted thiols, substituted thioesters, substituted esters as, among others, described in Handbook of Proteolytic Enzymes, Academic Press, 1998, Peters et al., Curr. Microbiol. 18, 1989; Harper et al., Anal. Biochem. 118, 1981; Janowski et al., Anal. Biochem. 252, 1997; Glucksman et al., Biophys. J. 62, 1992, or Soeda et al., Chem. Pharm. Bull. 33, 1985.

It is within the present invention that the tag or a first part of the tag is removable after the coupling to the first peptide, i.e. the tag or a first part thereof is removed, again, from construct comprising the tag or a first part of the tag. The advantage arising from this is that, a stoichiometric relationship between the tag, more specifically the removed tag, and the first peptide existing, the thus removed or released tag is an indication of the first peptide, more specifically the first peptide coupled to the tag. This allows the quantification of the first peptide and the first peptide coupled to the tag, prior to the removal of the tag, respectively, by quantifying the tag. Although both the first tag and the second tag may be used as the tag on which the quantification is based, it is preferred that the tag used for the quantification is the first tag.

The removal of the tag from the first peptide may occur by chemical cleavage, enzymatic cleavage and/or physical cleavage. The means for performing such chemical cleavage, enzymatic cleavage or physical cleavage are known to a person skilled in the art. In a preferred embodiment, such means are those described herein in connection with sequence-specific hydrolysis with which the C-terminal end of a peptide is generated. In an embodiment, the means used in the cleavage of the tag from the first peptide is the one which is used in the sequence-specific hydrolysis with which the C-terminal end of a peptide is generated. In a preferred embodiment, the removal of the first tag or a first part thereof is sequence-specific. This, preferably, follows from the use of said means, i.e. means for chemical cleavage, enzymatic cleavage and physical cleavage which, in such embodiment, are means for sequence-specific chemical cleavage, means for sequence-specific enzymatic cleavage or means for sequence-specific physical cleavage.

One of the functions of the tag in connection with the instant invention is to provide a signal which may be detected. Such signal can be provided by the tag, by the label attached to the tag, or both. Such signal is for use in a method for determining the presence and/or quantity of a target polypeptide of which a first peptide is a proteotypic peptide, in at least one mixture of different polypeptides making use of an internal peptide standard. Preferably such method is the method for determining the presence and/or quantity of a target polypeptide of which a first peptide is a proteotypic peptide, in at least one mixture of different polypeptides of the invention. In an embodiment, the signal is indicative of the tag and is even more preferably a quantitative signal. In another embodiment, the signal is proportionate to the tag or a part thereof. It is preferred that the signal follows a 1:1 stoichiometry, i.e. one signal unity corresponds to one tag or label. It will be understood by a person skilled in the art that also other stoichiometries may be used in the practicing of the methods of the invention as long as the stoichiometry is known and reproducible in the practicing of the method.

What has been said for the removal of the tag from the first by chemical cleavage, enzymatic cleavage and/or physical cleavage, in its diverse embodiments disclosed herein, applies or is applicable also to the embodiment where only a part of the tag is removed from the construct comprising both the tag and the first peptide. In this embodiment, thus, the tag is removed from the first peptide only in part. Consequently a part of the tag remains coupled to the first peptide, whereas another part of the tag is removed from the construct comprising the tag and the first peptide. A typical example for this embodiment are the so-called FRET systems where one of the two constituents of the FRET system is separated from the other constituent of the FRET system in the course of the cleaving off of a part of the tag from the construct comprising the first peptide and the tag. By such cleaving off, a signal is generated which may be detected and used in connection with the methods of the invention. FRET systems are, for example, described in Yaron et al. 1979, Bratovanova and Petkov 1987, Meldal et al. 1994, Wang et al. 1990, Lee et al., Blood 103, 204.

Depending on the signal, various detection methods may be used. Such detection methods may be selected from the group comprising fluorescence detection, UV detection and mass detection. It is within the skills of a person of the art to determine which detection method is used for which signal and this for which tag and label, respectively.

The tag may be used for the immobilization of the construct comprising the first peptide and the tag. In an embodiment, the tag is immobilized to a surface and subsequently the first peptide is coupled to the tag. In an embodiment, the tag is immobilized to a surface and the amino acids or groups of amino acids forming the amino acid sequence of the first peptide are coupled or attached to the immobilized tag or to the immobilized tag to which already one or several of the amino acids of the amino acid sequence of the first peptide has/have been coupled or attached. In an embodiment thereof the tag is the first tag. In an alternative embodiment, the tag is the second tag.

In an embodiment where the second tag is a protease substrate, it is preferred that the protease substrate is selected from the group comprising peptides and peptide derivatives mimicking the N-terminal part of endoprotease substrates. In an embodiment where the first tag is a protease substrate, which is preferably attached to the C-terminal and of the first peptide, it is preferred that the protease substrate is selected from the group comprising peptides and peptide derivatives mimicking the C-terminal part of endoprotease substrates.

In an embodiment, the second tag is a peptide. Preferably such peptide has one or several of the characteristics of the first tag being a peptide. In an embodiment, the peptide being or being part of the second tag, is attached to the N-terminal end, preferably to the N-terminal amino acid residue of the amino acid sequence of the first peptide.

In the embodiment where a first tag and a second tag are coupled to the first peptide, the first tag and the second tag are preferably different from each other. It is within the present invention that the first tag is coupled to the first peptide prior to the coupling of the second tag. It is, however, also within the present invention that the second tag is coupled to the first peptide prior to the coupling of the first tag. This applies equally in case the first and second tag, respectively, are not coupled as a full length molecule, but also in those embodiments where the building blocks of the first tag and second tag are sequentially attached to the first peptide.

In the embodiment where a first tag and a second tag are coupled to the first peptide, in an embodiment, the first tag and the second tag are different. In the embodiment where a first tag and a second tag are coupled to the first peptide and where the first tag and the second tag are different, the label of the first tag is different from the label of the second tag. Alternatively, the label of the first tag and the label of the second tag are identical.

In the embodiment where a first tag and a second tag are coupled to the first peptide, in an embodiment, the first tag and the second tag are identical, but the label of the first tag and the label of the second tag are different.

In an embodiment, the second tag comprises an anchor moiety. The anchor moiety is preferably attached to a linker, whereby the linker is arranged between the anchor moiety and the second tag. Preferably, the anchor moiety is suitable for attaching the second tag and/or the internal peptide standard to a surface which is preferably a solid surface. In an alternative embodiment, the surface is a non-solid surface such as polymers which are soluble but able to shrink and precipitate if the solvents are changed. Such polymers are known to the person skilled in the art (Reactive and Functional Polymers, Volume 44, Issue 1, 14 Apr. 2000, Pages 41-46, The effect of PEG groups on swelling properties of PEG-grafted-polystyrene resins in various solvents, Byeong-Deog Parka and Yoon-Sik Lee; BioPharm International Supplements, Mar. 2, 2010, PEG Precipitation: A Powerful Tool for Monoclonal Antibody Purification, Michael Kuczewski, Emily Schirmer, PhD, Blanca Lain, PhD, Gregory Zarbis-Papastoitsis, PhD; Current Opinion in Chemical Biology, Volume 1, Issue 1, June 1997, Pages 107-113, Synthesis on soluble polymers: new reactions and the construction of small molecules, Dennis J Gravert and Kim D Janda).

In an embodiment, such polymers are modified ethylene glycols. The attaching to a surface may be used for purification purposes of the internal peptide standard. In an embodiment, the anchor moiety allows the reversible attachment of the internal peptide standard to a surface. However, it is also within the present invention that the attachment of the anchor moiety and/or if the internal peptide standard is irreversible. In case of the embodiment where the attachment is irreversible, the attachment is a covalent attachment. In a preferred embodiment, the covalent attachment is established by means of a chemoselective reaction of the anchor moiety, the second tag or the first internal peptide standard.

In an embodiment the anchor moiety is selected from the group comprising biotin, iminobiotin, desthiobiotin, avidin, streptavidin, whereby the surface to which the anchor moiety binds, comprises an interaction partner of these anchor moieties which are known in the art to bind to each other. Alternatively, the anchor moiety is a chemical group allowing chemoselective reaction with the appropriate reactive function on the surface. Such chemical groups represent chemical groups which are not found in the first peptide and/or the first tag like maleimido groups; alpha-halo-ketones like bromopyruvic acid or 4-carboxy-alpha-Bromo-acetophenone, alpha-isothiocyanato-ketones like 4-carboxy-alpha-isothiocyanato-acetophenon, aldehydes like carboxybenzaldehyde, ketone like levulinic acid, thiosemicarbazides, thioamides like succinic monothioamide, alpha-bromo-carboxylic acids like bromo acetic acid, hydrazines like 4-hydrazinobenzoic acid, O-alkylhydroxylamines like Amino-oxy-acetic acid, and hydrazides like glutaric acid monohydrazide.

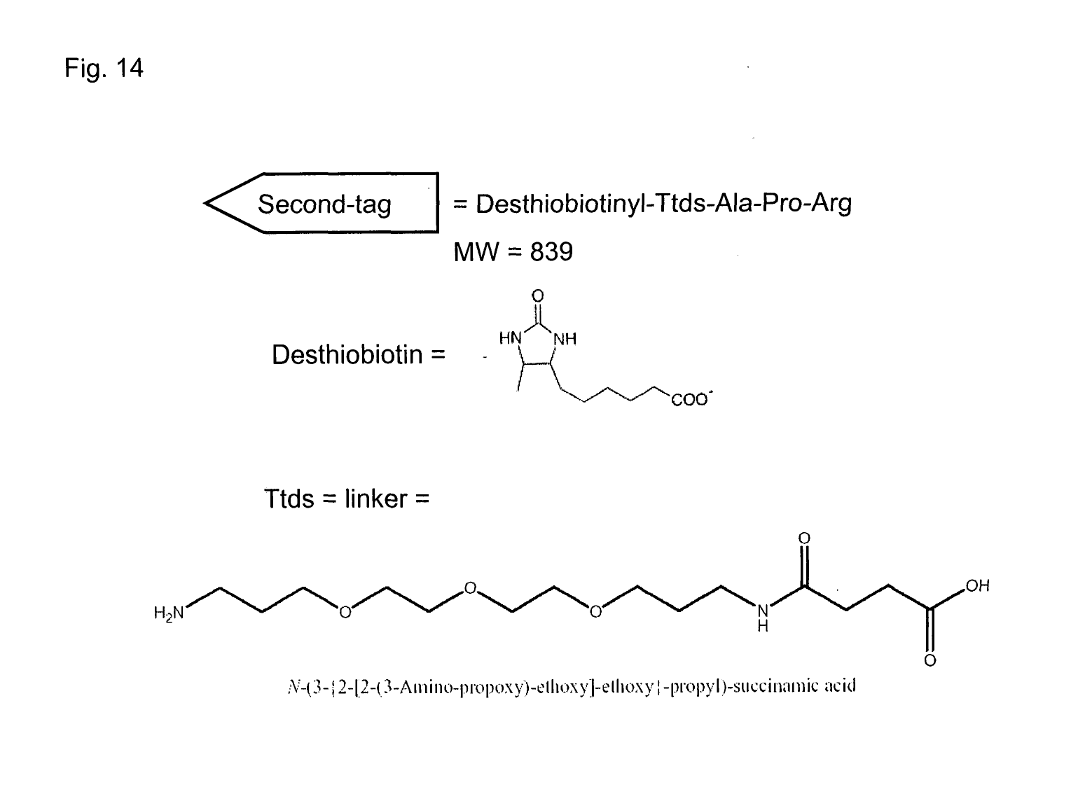

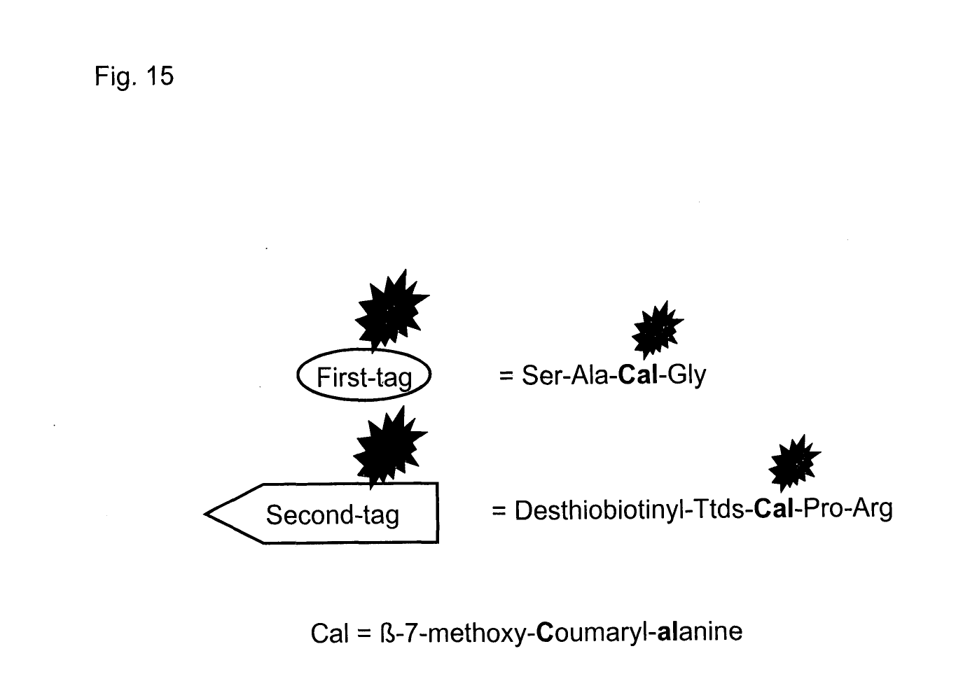

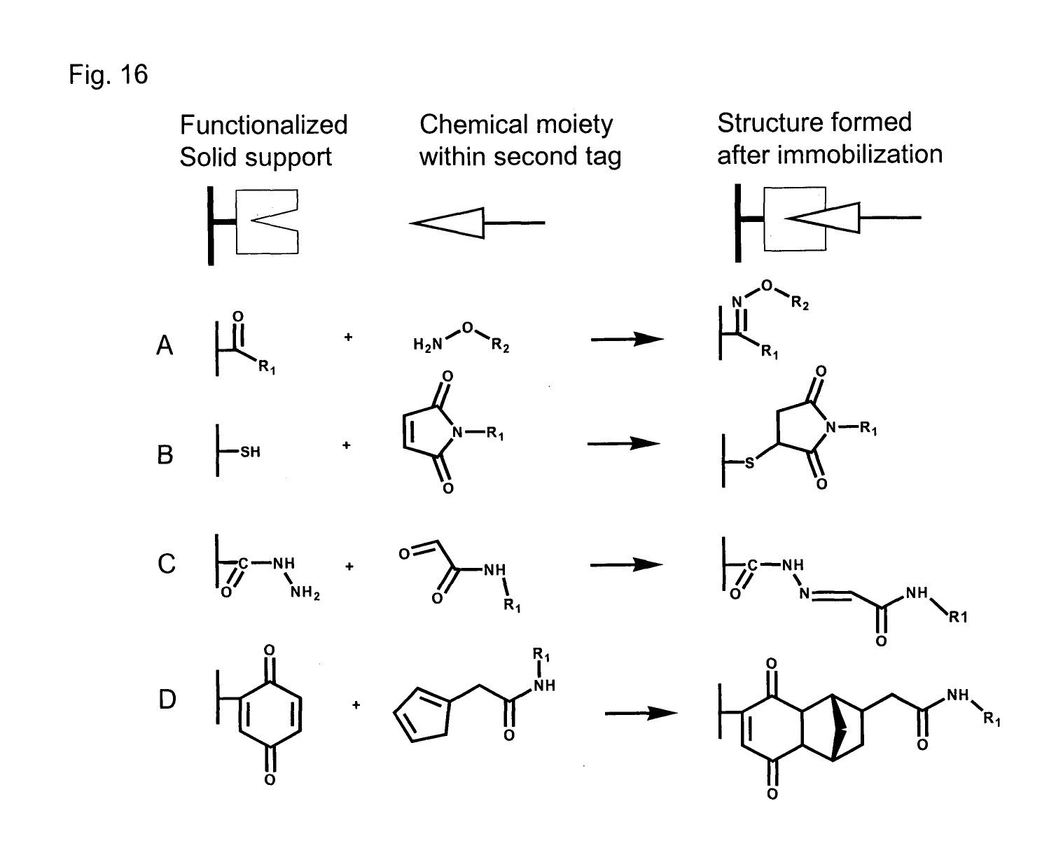

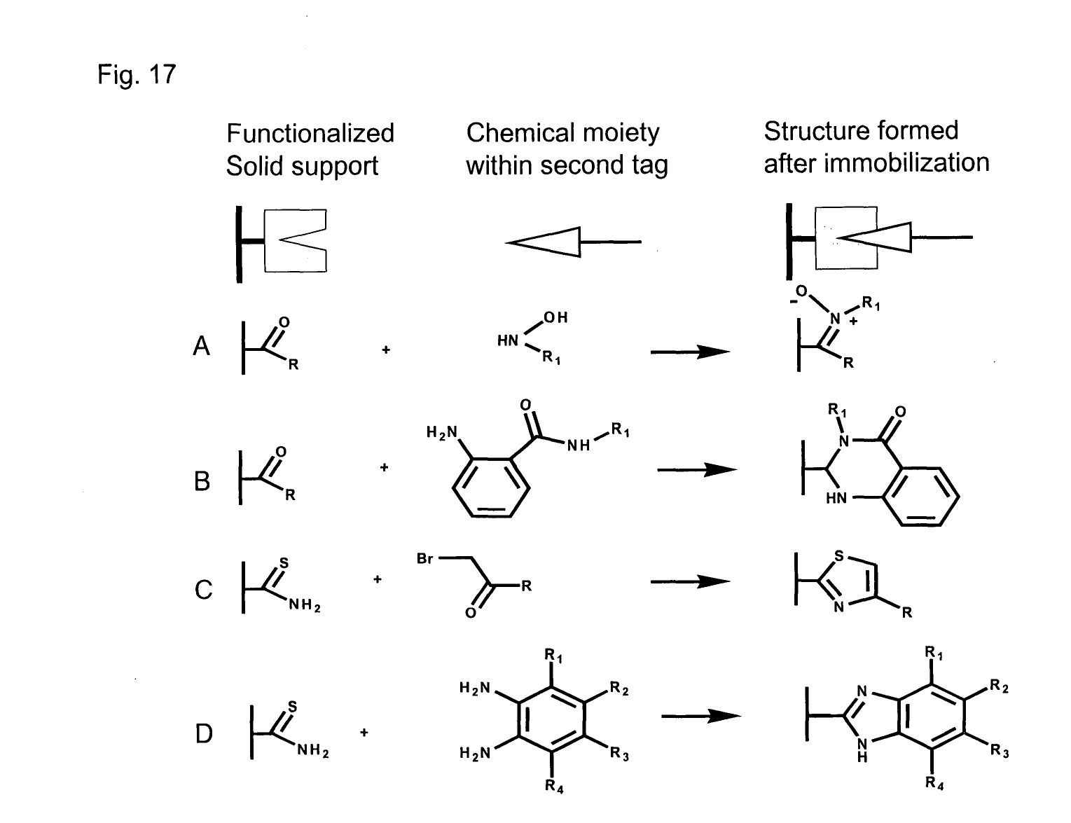

More specifically, a number of reactions known as such to the person skilled in the art can be used so as to effect a chemosselective reaction for the attachment of an anchor moiety or the second tag or the internal standard pepetide. Such reaction are described, among others, in Lemieux & Bertozzi (Lemieux, G. A. & Bertozzi, C. R., 1998, Chemoselective ligation reactions with proteins, oligosaccharides and cells, TIBTECH, 16, 506-513, see FIGS. 16 and 17 for). With a view to the required directional immobilisation it should basically be ensured that under the respective interaction conditions, substantially only one special compound is formed between the amino acid sequence and the surface (FIGS. 14 and 15). The choice of the reactive group on the amino acid sequence side will thus depend substantially on the individual sequence. Alternatively it is provided within the scope of the present invention that a terminal structure standard to all the amino acid sequences is provided and this terminal structure is made available for the specific reaction with the surface, especially an activated surface (FIGS. 14 and 15). Typically during the chemoselective reactions amino or carboxyl groups contained in the amino acid sequence are not adversely affected. Examples of suitable reactions are the formation of thioethers from halo-carbonic acids and thiols, which include the formation of thioethers from halocarbonic acids and thiols, thioethers from thiols and maleinimides, amide bonds from thioesters and 1,2-aminothiols, thioamide bonds from dithioesters and 1,2-aminothiols, thiazolidines from aldehydes and 1,2-aminothiols, oxazolidines from aldehydes/ketones and 1,2-amino alcohols, imidazoles from aldehydes/ketones and 1,2-diamines (see also FIGS. 16 and 17), thiazols from thioamides and alpha-halo-ketones, aminothiazols from amino-oxy-compounds and alpha-isothiocyanato-ketones, oximes from amino-oxy-compounds and aldehydes, oximes from amino-oxy-compounds and ketones, hydrazones from hydrazines and aldehydes, hydrazones from hydrazides and ketones. Moreover, the radicals R1-R5 shown in FIGS. 16 and 17 or the residues in the above-mentioned chemoselective reactions can be alkyl, alkenyl, alkynyl, cycloalkyl or aryl radicals or heterocyclic compounds, wherein alkyl stands for branched and unbranched C₁₋₂₀-alkyl, C₃₋₂₀-cycloalkyl, preferably for branched and unbranched-C₁₋₁₂ alkyl, C₃₋₁₂-cycloalkyl, and especially preferably for branched and unbranched C₁₋₆-alkyl, C₃₋₆-cycloalkyl radicals. Alkenyl stands for branched and unbranched C₂₋₂₀-alkenyl, branched and unbranched C₁₋₂₀-alkyl-O—C₂₋₂₀ alkenyl, C₁₋₂₀(—O/S—C₂₋₂₀)₂₋₂₀ alkenyl, aryl-C₂₋₂₀-alkenyl, branched and unbranched heterocyclyl C₂₋₂₀ alkenyl, C₃₋₂₀-cycloalkenyl, preferably for branched and unbranched C₂₋₁₂-alkenyl, branched and unbranched C₁₋₁₂(—O/S—C₂₋₁₂)₂₋₁₂ alkenyl, especially preferably for branched and unbranched C₂₋₆-alkenyl, branched and unbranched C₁₋₆(—O/S—C₂₋₈)₂₋₈ alkenyl radicals; alkynyl stands for branched and unbranched C₂₋₂₀-alkynyl, branched and unbranched C₁₋₂₀(—O/S—C₂₋₂₀)₂₋₂₀ alkynyl, preferably for branched and unbranched C₂₋₁₂-alkynyl, branched and unbranched C₁₋₁₂(—O/S—C₂₋₁₂)₂₋₁₂ alkynyl, especially preferably for branched and unbranched C₂₋₆-alkynyl, branched and unbranched C₁₋₆(—O/S—C₂₋₈)₂₋₈ alkynyl radicals; cycloalkyl stands for bridged and unbridged C₃₋₄₀-cycloalkyl, preferably for bridged and unbridged C₃₋₂₆-cycloalkyl, especially preferably for bridged and unbridged C₃₋₁₅-cycloalkyl radicals; aryl stands for substituted and unsubstituted mono- or multi-linked phenyl, pentalenyl, azulenyl, anthracenyl, indacenyl, acenaphtyl, fluorenyl, phenalenyl, phenanthrenyl, preferably for substituted and unsubstituted mono- or multi-linked phenyl, pentalenyl, azulenyl, anthracenyl, indenyl, indacenyl, acenaphtyl, fluorenyl, especially preferably for substituted and unsubstituted mono- or multi-linked phenyl, pentalenyl, anthracenyl radicals as well as their partly hydrated derivatives. Heterocyclic compounds can be unsaturated and saturated 3-15-membered mono-, bi- and tricyclic rings with 1-7 heteroatoms, preferably 3-10-membered mono-, bi- and tricyclic rings with 1-5 heteroatoms and especially preferably 5-, 6- and 10-membered mono-, bi- and tricyclic rings with 1-3 heteroatoms.

In addition, at the alkyl, alkenyl, alkynyl, cycloalkyl, aryl, heteroatoms, heterocyclic compounds, biomolecules or natural substance, 0 to 30 (preferably 0 to 10, especially preferably 0 to 5) of the following substituents can occur singly or in combination with one another: fluorine, chlorine, bromine, iodine, hydroxyl, amide, ester, acid, amine, acetal, ketal, thiol, ether, phosphate, sulphate, sulphoxide, peroxide, sulphonic acid, thioether, nitrile, urea, carbamate, wherein the following are preferred: fluorine, chlorine, bromine, hydroxyl, amide, ester, acid, amine, ether, phosphate, sulphate, sulphoxide, thioether, nitrile, urea, carbamate and especially preferred are: chlorine, hydroxyl, amide, ester, acid, ether, nitrile.

The linker which is arranged between the anchor moiety and the second tag, in an embodiment, the linker is a chemical structure composed of at least two functional groups. The linker preferably connects the anchor moiety with the first peptide in a covalent manner. The linker spaces the anchor moiety apart from the first peptide allowing the proper interaction of the anchor moiety with the solid surface. Additionally, the linker enables efficient enzymatic cleavage of the second tag/first peptide-bond.

In an embodiment, the linker is selected from the group comprising N-(3-{2-[2-(3-amino-propoxy)-ethoxy]-ethoxy}-propyl)-succinamic acid or alkanes either branched or linear chains, preferably those composed of 2-30 carbon atoms, more preferably those composed of 4-20 carbon atoms and most preferably those composed of 4-10 carbon atoms; or polyethers, that means polymers of ethyleneoxide or propylene oxide, preferably polymers composed of 1-10, more preferably 2-5 ethylenoxide or polypropylene oxide units, or polyalcohols, branched or unbranched, like polyglycole and derivatived thereof like O,O′-bis(2-aminopropyl)-polyethylenglycol or polyurethanes, polyhydroxy acids, polycarbonates, polyimides, polyamides, polyester, polysulfones, preferably those composed of 1-100 monomeric units, more preferably those composed of 1-50 monomeric units and most preferably those composed of 1-20 monomeric units, or combinations of the mentioned alkanes with the mentioned polyurethanes, polyhydroxy acids, polycarbonates, polyimides, polyamides, polyester, polysulfones, or diaminoalkanes either branched or linear chains, preferably those composed of 2-30 carbon atoms, more preferably those composed of 2-20 carbon atoms and most preferably those composed of 2-10 carbon atoms like 1,3-diaminopropane, 1,6-diaminohexane and 1,8-diaminooctane, as well as combinations of diaminoalkanes with polyethers, like 1,4-bis-(3-aminopropoxy)butan or carboxylic acids and dicarboxylic acids and derivatives like hydroxy-, mercapto- and amino carboxylic or dicarboxylic acids with either branched or linear chains, preferably those composed of 2-30 carbon atoms, more preferably those composed of 2-20 carbon atoms and most preferably those composed of 2-10 carbon atoms like succinic acid or glutaric acid. Amino acids and peptides preferably composed of 1-20 residues or more preferably 1-10 residues or most preferably 1-3 residues like trimers of lysine, dimmers of 3-aminopropionic acid and monomers of 6-aminohexanoic acid. Preferably the linker is a combination of a dicarboxylic acid with a diamino polyether like N-(3-{2-[2-(3-amino-propoxy)-ethoxy]-ethoxy}-propyl)-succinamic acid.

In an embodiment the second tag comprises a peptide comprising an amino acid sequence, whereby the linker is arranged between the anchor moiety and the N-terminal end, preferably between the N-terminal amino acid residue of the second tag.

In an embodiment of the method of the invention, the internal peptide standard comprises a second tag, a first peptide and a first tag, wherein the internal peptide standard is contained in a mixture of peptides, wherein the internal peptide standard is immobilized to a surface, and wherein the internal peptide standard specifically interacts with the surface through the anchor moiety of the internal peptide standard. In one or several subsequent steps, the peptides which are different from the internal peptide standard are removed from the mixture. Preferably, the specificity of the immobilization of the internal peptide standard by means of the second tag and the anchor moiety, respectively, allows for the interaction of the second tag and the anchor moiety, respectively, with the surface. Because of this, any non-binding or non-specifically binding compounds can be removed such as by washing, which results in purification of the intended internal peptide standard, typically immobilized to the surface.

From the above it will be acknowledged that, in an embodiment, the internal peptide standard is attached to a surface. In case the attachment is reversible such as by the use of biotin, desthiobiotin, avidin or streptavidin, the addition of these and similar compounds will result in a competition between the thus immobilized internal peptide standard and the interaction partner of said compounds, whereupon the immobilized internal peptide standard will be released. In an alternative embodiment where the attachment is covalent, the use of a means for sequence-specific cleavage between the second tag and the first peptide, whether or not comprising a first tag, is preferred whereupon such first peptide will be released from the surface. In any case, this kind of procedure provides for a purified internal peptide standard.

The thus obtained or any form of purified internal peptide standard may be further used, preferably in any method of the invention, including and in particular the methods for preparing a library of internal peptide standards, wherein the library consists of a plurality of species of an internal peptide standard, and the methods for determining the presence and/or quantity of a target polypeptide of which a first peptide is a proteotypic peptide, in at least one mixture of different polypeptides. For use in said methods, the thus purified internal peptide standard, regardless whether it still contains the second and/or the first tag, is preferably transferred to a different reaction vessel. It will be acknowledged by a person skilled in the art that depending on the various embodiments of said methods, the second tag is removed prior to the used of the internal peptide standard. It will also be acknowledged by a person skilled in the art that depending on the various embodiments of said methods, the first tag is removed prior to the used of the internal peptide standard. Finally, the will be acknowledged by a person skilled in the art that depending on the various embodiments of said methods, the first and the second tag are removed prior to the used of the internal peptide standard. With the removal of the first tag, the second tag or both the first tag and the second tag, there might go along a method for the quantification of the first, second or both the first and the second tag and thus, ultimately, of the internal peptide standard. Methods for the quantification of the first tag, the second tag and the first and the second tag are known to a person skilled in the art. The specific method used depends on the kind of the individual tag and, in the embodiments where the tag bears at least one label, on the kind of label as will be acknowledged by and is evident for a person skilled in the art.

The present invention is also related to a library or panel of internal peptide standards and a method for preparing the same. Preferably, the library or panel of internal peptide standards comprises a plurality of species of an internal peptide standard. As preferably used herein, a plurality of species of internal peptide standard means at least two internal peptide standards. The method of the invention for preparing a library of internal peptide standards comprises the following steps:
a) individually preparing a plurality of species of an internal peptide standard, whereby each species of an internal peptide standard is prepared according to the method for preparing an internal peptide standard of the invention; and
b) optionally mixing the individually prepared species of an internal peptide standard,
   wherein the species of an internal peptide standard differ from each other in the first and/or second tag and/or the amino acid sequence of first peptide.

In an embodiment, the first and/or the second tag comprises a peptide having an amino acid sequence and the amino acid sequence of each species of the library of internal peptide standards is different from the amino acid sequence of the other species of the library of internal peptide standards. In an embodiment thereof, the amino acid sequence of the various species differs from each other with regard to the amino acid sequence of the first peptide.

In the embodiment where the amino acid sequence of the various species differs from each other with regard to the amino acid sequence of the first peptide, the first and the second tag of the various species may be the same. If the first and the second tag of the various species are the same and if they bear a label, it is within the invention that the label is the same for the various first and/or second tags of the various species. In such case, the quantification of the label typically provides information on the overall number of internal peptide standard molecules contained in the reaction or vessel, not discriminating between the individual numbers of molecules contributed by the individual various species. If the first and the second tag of the various species are the same and if they bear a label, it is within the invention that the labels of the first tag and/or the second tag of the various species are different; more preferably the label for the first tag of the various species is different and thus characteristic for the individual species. In such case, the quantification of the labels typically provides information on the number of the various individual internal peptide standards contained in the reaction or vessel, discriminating between the individual numbers of molecules contributed by the individual various species. The same considerations apply in case the first tag(s) and the second tag(s) do not require a specific label but that the first tag(s) and the second tag(s) due to their chemical and/or physical characteristics provide the information otherwise conferred by the label. One example for such a case which thus constitutes and embodiment, is the combinatorial use of defined amino acid residues for generation of the sequence(s) of the first and/or the second tag(s) in such a way that the sequence(s) of the first and/or the second tag represent a code for the sequence of the respective first peptide. In other words, the specific first and/or the specific second tag are indicative of a or the specific first peptide and the identification and/or the quantification of the specific first and/or second tag is factually the identification and/or the quantification of the first peptide. Therefore, if there is a second specific first and/or second specific second tag which are indicative of a second specific first peptide, in the method of the invention for determining the presence and/or quantity of a target polypeptide of which a first peptide is a proteotypic peptide, one may discriminate between the said first peptide and said second first peptide in the mixture of different polypeptides, under the proviso that the second first peptide is a proteotypic peptide of a target polypeptide, preferably a second target polypeptide.

In a second aspect, the present invention is related to methods for determining the presence and/or quantity of a target polypeptide of which a first peptide is a proteotypic peptide, in at least one mixture of different polypeptides.

In a first basic embodiment the method for determining the presence and/or quantity of a target polypeptide of which a first peptide is a proteotypic peptide, in at least one mixture of different polypeptides of the invention, is a method for determining the presence and/or quantity of a target polypeptide of which a first peptide is a proteotypic peptide, in at least one mixture of different polypeptides, comprising the following steps:
a) providing a mixture of different polypeptides preferably containing the target polypeptide;
b) adding an internal peptide standard of the invention or of a panel of internal peptide standards of the invention, whereby preferably the amount of the internal peptide standard of the invention or of the panel of internal peptide standards of the invention is unknown, thereby generating a spiked mixture;
c) treating the spiked mixture with a means for sequence-specific hydrolysis to generate a plurality of peptides from the mixture of different polypeptides and to cleave off the first tag and/or the second tag from the internal peptide standard/standards thus releasing the first peptide, whereby the plurality of peptides comprises the proteotypic peptide derived from the different polypeptides and the first peptide released from the internal standard;
d) determining the amount of the first and/or second tag contained in the spiked mixture and therefrom the quantity of the internal standard/standards added in step b);
e) determining the ratio of the first peptide to the proteotypic peptide derived from the different polypeptides, each generated in step c); and
f) calculating from the ratio and the quantity of the internal standard/standards determined in step d) the quantity of the target polypeptide in the mixture of different polypeptides,
   wherein either the proteotypic peptide derived from the different polypeptides or the first peptide of the internal peptide standard/standards comprise a mass label. It will be acknowledged by a person skilled in the art that a mixture of different polypeptides preferably containing the target polypeptide is added to a quantity of an internal peptide standard of the invention or of a panel of internal peptide standards of the invention, thereby generating a spiked mixture.

In this first basic embodiment, the quantity of the internal peptide standard of the invention is not known when such internal peptide standard is added to the mixture of different polypeptides. The first tag and/or the second tag are only cleaved off from the first peptide when the proteotypic peptide is generated from the mixture of different polypeptides by means of sequence-specific hydrolysis for which the means for sequence-specific hydrolysis as, e.g., disclosed herein, may be used. In this first basic embodiment it is preferred that the sequence-specific hydrolysis resulting in the release of the proteotypic peptide and the release of either or both of the first and the second tag, preferably the first tag, is effected by the same means for sequence-specific hydrolysis.

In the first basic embodiment, the spiked mixture after having been treated with a means for sequence-specific hydrolysis in step c) may be subject to a fractionation step. Such fractionation step may be performed by means of a chromatographic procedure, preferably by liquid chromatography. The purpose of such fractionation step is to reduce the complexity of the reaction mixture obtained upon having performed step c). It will be acknowledged by a person skilled in the art that in such step c) a huge number of peptides may be generated by sequence-specific hydrolysis of the mixture of different polypeptides, depending on the number of species of polypeptides contained in said mixture. In complex mixtures such as a biological sample, more specifically a lysate of a cell or organ, the sequence-specific hydrolysis may ultimately result in the generation of hundreds of thousands of peptides. By reducing the complexity of the reaction mixture obtained upon having performed step c) the step of determining the amount of the first and/or second tag contained in the spiked mixture and determining therefrom the quantity of the internal standard/standards added in step b), and more preferably the determining of the ratio of the first peptide to the proteotypic peptide derived from the different polypeptides as subject to step e) can be performed more easily and the performance of the analytical device can be less advanced.

In an embodiment of the method according to the first basic embodiment after step c) and prior to step e) the treated spiked mixture, i.e. the reaction mixture obtained after having performed step c), is subjected to a separation step where the first peptide and the proteotypic peptide derived from the different polypeptides are separated from components of the treated spiked mixture. This embodiment is factually a very specific embodiment of the immediately preceding embodiment.

In an embodiment of the method according to the first basic embodiment after step c) and prior to step d) the treated spiked mixture is subjected to a separation step where the first and the second tag is/are separated from components of the treated spiked mixture. By this separation step, first and/or the second tag are removed from the reaction mixture obtained after having performed step c). Such separation of the first tag and/or the second tag can be advantageous insofar that the detection and quantification of the first and/or second tag is not obstructed or obscured by the other compounds contained in the reaction mixture obtained after having performed step c). This allows for a higher accuracy and/or a less complex analytical method for the detection and quantification of the first and/or the second tag.

In a second basic embodiment the method for determining the presence and/or quantity of a target polypeptide of which a first peptide is a proteotypic peptide, in at least one mixture of different polypeptides of the invention, is a method for determining the presence and/or quantity of a target polypeptide of which a first peptide is a proteotypic peptide, in at least one mixture of different polypeptides, comprising:

a) providing a mixture of different polypeptides preferably containing the target polypeptide;
b) providing an internal peptide standard of the invention or a panel of internal peptide standards of the invention, whereby preferably the provided amount of the internal peptide standard and/or of the panel of internal peptide standards is unknown, treating the internal peptide standard or the panel of internal peptide standard with a means for sequence-specific hydrolysis to cleave off the first tag and/or the second tag from the internal peptide standard or the panel of internal peptide standards thus releasing the first peptide;
either
ca1) adding the reaction mixture obtained in the performance of step b) to the mixture of different polypeptides of step a) or adding the mixture of different polypeptides of step a) to reaction mixture obtained in the performance of step b) and
ca2) treating the mixture obtained in the performance of step ca1) with a means for sequence-specific hydrolysis to generate a plurality of peptides from the mixture of different polypeptides, whereby the plurality of peptides comprises the proteotypic peptide;
or
cb1) treating the mixture of different polypeptides of step a) with a means for sequence-specific hydrolysis to generate a plurality of peptides from the mixture of different polypeptides, whereby the plurality of peptides comprises the proteotypic peptide; and
cb2) adding the reaction mixture obtained in the performance of step b) to the mixture obtained in the performance of the step cb1) or adding to the mixture obtained in the performance of the step cb1) to the reaction mixture obtained in the performance of step b)
whereby after step ca2) and cb2), respectively, a spiked mixture is obtained;
d) determining the amount of the first and/or second tag contained in the spiked mixture and therefrom the quantity of the internal standard/standards added in step b);
e) determining in the spiked mixture the ratio of the first peptide to the proteotypic peptide derived from the different polypeptides; and
f) calculating from the ratio and the quantity of the internal standard/standards determined in step d) the quantity of the target polypeptide in the mixture of different polypeptides,
wherein either the proteotypic peptide derived from the different polypeptides or the first peptide of the internal peptide standard/standards comprise a mass label.

In this second basic embodiment two basic alternatives are possible. In a first alternative, steps ca1) and ca2) are performed. In a second alternative, steps cb1) and cb2) are performed. Irrespective thereof, in this second embodiment the treatment of the internal peptide standard(s) with a means for sequence-specific hydrolysis is effected separately from the treatment of the mixture of different polypeptides preferably containing the target polypeptide.

In the second basic embodiment, the reaction mixture obtained after the performing of step ca2), or after the performing of step cb1) or cb2) may be subject to a fractionation step. Such fractionation step may be performed by means of a chromatographic procedure, preferably by liquid chromatography. The purpose of such fractionation step is to reduce the complexity of the reaction mixture obtained upon having performed step ca2), or the reaction mixture obtained after having performed step cb1) or cb2). It will be acknowledged by a person skilled in the art that in said steps ca2), cb1) and cb2) a huge number of peptides may be generated by sequence-specific hydrolysis of the mixture of different polypeptides, depending on the number of species of polypeptides contained in said mixture. In complex mixtures such as a biological sample, more specifically a lysate of a cell or organ, the sequence-specific hydrolysis may ultimately result in the generation of hundreds of thousands of peptides. By reducing the complexity of the reaction mixture obtained upon having performed steps ca2), cb1) and cb2), respectively, the step of determining the amount of the first and/or second tag contained in the spiked mixture and determining therefrom the quantity of the added internal standard/standards, and more preferably the determining of the ratio of the first peptide to the proteotypic peptide derived from the different polypeptides as subject to step e) can be performed more easily and the performance of the analytical device can be less advanced. In connection with this embodiment, the spiked mixture is also referring to the reaction mixture obtained after the performing of steps ca2) and cb2) and which has been subject to said fractionation step.

In an embodiment of the method according to the second basic embodiment prior to step e) the treated spiked mixture, i.e. the reaction mixture obtained after having performed step ca2), cb1) or cb2) is subjected to a separation step where the first peptide and the proteotypic peptide derived from the different polypeptides are separated from components of the treated spiked mixture. This embodiment is factually a very specific embodiment of the immediately preceding embodiment. In connection with this embodiment, the spiked mixture is also referring to the reaction mixture obtained after the performing of steps ca2) and cb2) and which has been subject to said separation step.

In an embodiment of the method according to the second basic embodiment prior to step d) the treated spiked mixture is subjected to a separation step where the first and the second tag is/are separated from components of the treated spiked mixture. By this separation step, first and/or the second tag are removed from the reaction mixture obtained after having performed step c). Such separation of the first tag and/or the second tag can be advantageous insofar that the detection and quantification of the first and/or second tag is not obstructed or obscured by the other compounds contained in the reaction mixture obtained after having performed steps ca2), cb1) an dcb2). This allows for a higher accuracy and/or a less complex analytical method for the detection and quantification of the first and/or the second tag. In connection with this embodiment, the spiked mixture is also referring to the reaction mixture obtained after the performing of steps ca2) and cb2) and which has been subject to said separation step.

In an embodiment of the method according to the second basic embodiment the means for sequence-specific hydrolysis used in step b) is removed from the mixture obtained in the performance of step b) prior to performing step ca1) or cb1). Such means are known to a person skilled in the art. Non-limiting examples for procedures for the removal of the means for sequence-specific hydrolysis are liquid chromatography, high performance reverse phase liquid chromatography, size exclusion chromatography, electrophoresis, capillary electrophoresis, gel electrophoresis and affinity chromatography. As used in this embodiment the removal of the means for sequence-specific hydrolysis also encompasses the inactivation of such means. For example, the means is an enzyme the enzyme may be inactivated by heat treatment, by pH shift or addition of an inhibitor to the enzyme activity.

In a third basic embodiment the method for determining the presence and/or quantity of a target polypeptide of which a first peptide is a proteotypic peptide, in at least one mixture of different polypeptides of the invention, is a method for determining the presence and/or quantity of a target polypeptide of which a first peptide is a proteotypic peptide, in at least one mixture of different polypeptides, comprising:

a) providing an internal peptide standard of the invention or a panel of internal peptide standards of the invention, whereby the amount of the internal peptide standard of the invention or of the panel of internal peptide standards of the invention is unknown, treating the internal peptide standard or the panel of internal peptide standards with a means for sequence-specific hydrolysis to cleave off the first tag and/or the second tag from the internal peptide standard or the panel of internal peptide standards thus releasing the first peptide;
b) optionally removing the means for sequence-specific hydrolysis;
c) determining the amount of the first and/or second tag contained in the mixture obtained from the performance of step a) of b) and therefrom the amount of the internal standard contained in said mixture;
d) providing a mixture of different polypeptides preferably containing the target polypeptide;
either
da1) adding a part of the reaction mixture obtained in the performance of step a) or b) to the mixture of different polypeptides of step d) or adding the mixture of different polypeptides of step d) to a part of the reaction mixture obtained in the performance of step a) or b) and
da2) treating the mixture obtained in the performance of step da1) with a means for sequence-specific hydrolysis to generate a plurality of peptides from the mixture of different polypeptides, whereby the plurality of peptides comprises the proteotypic peptide;
or
db1) treating the mixture of different polypeptides of step d) with a means for sequence-specific hydrolysis to generate a plurality of peptides from the mixture of different polypeptides, whereby the plurality of peptides comprises the proteotypic peptide; and
db2) adding a part of the mixture obtained in the performance of step a) or b) to the mixture obtained in the performance of the step db1) or adding the mixture obtained in the performance of the step db1) to adding a part of the mixture obtained in the performance of step a) or b);
whereby after step da2) and db2), respectively, a spiked mixture is obtained;
e) determining in the spiked mixture the ratio of the first peptide to the proteotypic peptide derived from the different polypeptides; and
f) calculating from the ratio and the quantity of the internal standard/standards the quantity of the target polypeptide in the mixture of different polypeptides,
wherein either the proteotypic peptide derived from the different polypeptides or the first peptide of the internal peptide standard/standards comprise a mass label.

In this third basic embodiment two basic alternatives are possible. In a first alternative, steps da1) and da2) are performed. In a second alternative, steps db1) and db2) are performed. Irrespective thereof, in this second embodiment the treatment of the internal peptide standard(s) with a means for sequence-specific hydrolysis is effected separately from the treatment of the mixture of different polypeptides preferably containing the target polypeptide and, additionally, as step c), the amount of the first and/or second tag contained in the mixture obtained from the performance of step a) of b) is determined and, therefrom, the amount of the internal standard contained in said mixture is determined. In accordance therewith, with the adding of a part of the reaction the reaction mixture obtained in the performance of step a) or b), preferably by means of a known aliquot of said reaction mixture, a known amount of the internal peptide standard is added to the mixture of different polypeptides preferably containing the target polypeptide, regardless whether such mixture of different polypeptides preferably containing the target polypeptide has at that stage of the method been subject to sequence-specific hydrolysis.

In the third basic embodiment, the reaction mixture obtained after the performing of step da2), or after the performing of step db1) or db2) may be subject to a fractionation step. Such fractionation step may be performed by means of a chromatographic procedure, preferably by liquid chromatography. The purpose of such fractionation step is to reduce the complexity of the reaction mixture obtained upon having performed step da2), or the reaction mixture obtained after having performed step db1) or db2). It will be acknowledged by a person skilled in the art that in said steps da2), db1) and b2) a huge number of peptides may be generated by sequence-specific hydrolysis of the mixture of different polypeptides, depending on the number of species of polypeptides contained in said mixture. In complex mixtures such as a biological sample, more specifically a lysate of a cell or organ, the sequence-specific hydrolysis may ultimately result in the generation of hundreds of thousands of peptides. By reducing the complexity of the reaction mixture obtained upon having performed steps da2), db1) and db2), respectively, the step of determining of the ratio of the first peptide to the proteotypic peptide derived from the different polypeptides as subject to step e) can be performed more easily and the performance of the analytical device can be less advanced. Particularly in case the ratio of the first peptide to the proteotypic peptide derived from the different polypeptides is determined by mass spectrometry, a mass spectrometer can be used having a lower resolution which allows that the methods of the invention can be used also by laboratory not having the most sophisticated mass spectrometers at their disposal. In connection with this embodiment, the spiked mixture is also referring to the reaction mixture which has been subject to said fractionation step and which is obtained after the performing of steps da2) and db2).

In an embodiment of the method according to the third basic embodiment prior to step e) the treated spiked mixture, i.e. the reaction mixture obtained after having performed step da2), db1) or db2) is subjected to a separation step where the first peptide and the proteotypic peptide derived from the different polypeptides are separated from components of the treated spiked mixture. This embodiment is factually a very specific embodiment of the immediately preceding embodiment. In connection with this embodiment, the spiked mixture is also referring to the reaction mixture which has been subject to said fractionation step and which is obtained after the performing of steps da2) and db2).

In an embodiment of the method according to the third embodiment prior to step da1) the reaction mixture obtained in the performance of step a) or b) is subjected to a separation step where the first and the second tag is/are separated from components of said reaction mixture. Such separation of the first tag and/or the second tag can be advantageous insofar that the detection and quantification of the first and/or second tag is not obstructed or obscured by the other compounds contained in the reaction mixture obtained after having performed steps da2), db1) and db2). This allows for a higher accuracy and/or a less complex analytical method for the detection and quantification of the first and/or the second tag. In connection with this embodiment, the spiked mixture is also referring to the reaction mixture obtained after the performing of steps da2), db1) and cb2) and which has been subject to said separation step. In an alternative embodiment of the method according to the third embodiment prior to step db2) the reaction mixture obtained in the performance of step db1) is subjected to a separation step where the proteotypic peptide derived from the different polypeptides is separated from components of said reaction mixture. This embodiment is factually a very specific embodiment of the above described embodiment comprising the fractionation step. In connection with this embodiment, the spiked mixture is also referring to the reaction mixture obtained after the performing of steps da2), db1) and db2) and which has been subject to said separation and/or fractionation step.

In an embodiment of the method according to the third embodiment the means for sequence-specific hydrolysis used in step a) is removed from the mixture obtained in the performance of step a) prior to performing step c) or d). In a further embodiment of the method according to the third basic embodiment the means for sequence-specific hydrolysis upon having been used in steps da1) or in step db1) is removed prior to performing step e). Such means are known to a person skilled in the art. Non-limiting examples for procedures for the removal of the means for sequence-specific hydrolysis are liquid chromatography, high performance reverse phase liquid chromatography, size exclusion chromatography, electrophoresis, capillary electrophoresis, gel electrophoresis and affinity chromatography. As used in this embodiment the removal of the means for sequence-specific hydrolysis also encompasses the inactivation of such means. For example, the means is an enzyme the enzyme may be inactivated by heat treatment or addition of an inhibitor to the enzyme activity.

In each and any of the first, second and third basic embodiment of the method of the invention for determining the presence and/or quantity of a target polypeptide of which a first peptide is a proteotypic peptide, in at least one mixture of different polypeptides, preferably either the proteotypic peptide derived from the different polypeptides or the first peptide of the internal peptide standard/standards comprise at least one mass label. The use of the mass label allows determining the ratio of the first peptide to the proteotypic peptide derived from the different polypeptides.

In connection with the methods of the invention for determining the presence and/or quantity of a target polypeptide of which a first peptide is a proteotypic peptide, in at least one mixture of different polypeptides, the ratio of the first peptide released from the internal peptide standard to the proteotypic peptide derived from the different polypeptides is preferably determined by mass spectrometry.

In each and any of the first, second and third basic embodiment of the method of the invention for determining the presence and/or quantity of a target polypeptide of which a first peptide is a proteotypic peptide, in at least one mixture of different polypeptides, the mass label is preferably selected from the group comprising C isotopes and N isotopes. Suitable C isotopes and N isotopes are known to the person skilled in the art. Preferred C isotopes are those which allow distinction of the first tag and/or the second tag and/or the first peptide and/or the proteotpyic peptide, preferably the distinction of the first peptide from the proteotypic peptide, by mass spectrometry. Such preferred C isotopes are preferably selected from the group comprising ¹³C isotopes. Preferred N isotopes are those which allow distinction of the first tag and/or the second tag and/or the first peptide and/or the proteotpyic peptide, preferably the distinction of the first peptide from the proteotypic peptide, by mass spectrometry. Such preferred N isotopes are preferably selected from the group comprising ¹⁵N isotopes. In this embodiment the internal peptide standard may contain a heavier or lighter isotope compared to the isotope of the mixture of different polypeptides preferably containing the target polypeptide and the proteotypic peptide, respectively. However, it is also within the present invention that the mixture of different polypeptides preferably containing the target polypeptide and the proteotypic peptide, respectively, may contain a heavier or lighter isotope compared to the isotope contained in the internal peptide standard. Such isotopes can be incorporated into the internal peptide standard by the use of building blocks of such internal peptide standard which contains the respective isotope(s), and/or such isotopes can be incorporated into the different polypeptides of the mixture of different polypeptides preferably containing the target polypeptide and the proteotypic peptide, respectively, by the use of building blocks of such different polypeptides and the proteotypic peptide bearing such isotope(s). In case the different polypeptides or the mixture of different polypeptides is derived from a biological system such as a cell, tissue, organ or organism the respectively labelled different polypeptides and proteotypic peptide can be obtained by feeding respectively labelled building blocks or precursors of such different polypeptides and proteotypic peptide to such biological system.

Due this difference in molecular mass arising from the use of differently labelled first peptide and proteotypic peptide, it is possible to determine the ratio of the first peptide to the proteotypic peptide derived from the different polypeptides, preferably by mass spectrometry.

Mass spectrometry as used in each and any of the first, second and third basic embodiment of the method of the invention for determining the presence and/or quantity of a target polypeptide of which a first peptide is a proteotypic peptide, in at least one mixture of different polypeptides, is known to a person skilled in the art and, for example, described in Gsteiger and Aebersold, Nat. Rev. Genet. 10, 2009 In a preferred embodiment, the mass spectrometry is multistage mass spectrometry. Pan et al., J. Proteome Res. 8, 2009.

In each and any of the first, second and third basic embodiment of the method of the invention for determining the presence and/or quantity of a target polypeptide of which a first peptide is a proteotypic peptide, in at least one mixture of different polypeptides, a peptide signature obtained by fragmentation which is diagnostic for the presence of the peptide is preferably known for the proteotypic peptide and thus for the first peptide, too. In an embodiment thereof, the peptide signature obtained by fragmentation is subject to subsequent analysis, preferably in a tandem mass spectrometer. Methods for the fragmentation are known to a person skilled in the art and are selected from the group comprising collision induced dissociation (see, e.g., Wells J M, McLuckey S A (2005). “Collision-induced dissociation (CID) of peptides and proteins”. Meth. Enzymol. 402: 148-85), electron capture dissociation (see, e.g., Zubarev R A, Kelleher N L, McLafferty F W (1998). “Electron capture dissociation of multiply charged protein cations. A nonergodic process”. J. Am. Chem. Soc. 120 (13): 3265-66.), electron transfer dissociation (see, e.g., Syka J E, Coon J J, Schroeder M J, Shabanowitz J, Hunt D F (2004). “Peptide and protein sequence analysis by electron transfer dissociation mass spectrometry”. Proc. Natl. Acad. Sci. U.S.A. 101 (26): 9528-33), infrared multiphoton dissociation (see, e.g., Little D P, Speir J P, Senko M W, O'Connor P B, McLafferty F W (1994). “Infrared multiphoton dissociation of large multiply charged ions for biomolecule sequencing”. Anal. Chem. 66 (18): 2809-15, blackbody infrared radiative dissociation (see, e.g., Schnier P D, Price W D, Jockusch R A, Williams E R (1996). “Blackbody Infrared Radiative Dissociation of Bradykinin and Its Analogues: Energetics, Dynamics, and Evidence for Salt-Bridge Structures in the Gas Phase”. Journal of the American Chemical Society 118 (30): 7178-7189).

In each and any of the first, second and third basic embodiment of the method of the invention for determining the presence and/or quantity of a target polypeptide of which a first peptide is a proteotypic peptide, in at least one mixture of different polypeptides, the internal peptide standard of the invention or the panel of internal standards of the invention is purified prior to the use in said method of the invention for determining the presence and/or quantity of a target polypeptide of which a first peptide is a proteotypic peptide, in at least one mixture of different polypeptides. In a preferred embodiment, the purification is based on or makes use of the second tag. In such embodiment the by-products of the synthesis of the internal peptide standard or of the panel of internal peptide standards are preferably removed from the reaction mixture while or with the internal peptide standard or the panel of internal peptide standard being immobilized or attached to a surface as also disclosed herein. In an alternative embodiment the internal peptide standard of the invention or the panel of internal peptide standards of the invention is not purified prior to the use in said method of the invention for determining the presence and/or quantity of a target polypeptide of which a first peptide is a proteotypic peptide, in at least one mixture of different polypeptides. In such alternative embodiment, the internal peptide standard of the invention or the panel of internal peptide standards of the invention may be used as obtained from the method of the invention for preparing an internal peptide standard.

In each and any of the first, second and third basic embodiment of the method of the invention for determining the presence and/or quantity of a target polypeptide of which a first peptide is a proteotypic peptide, in at least one mixture of different polypeptides, the amount of the internal peptide standard or of the panel of internal peptide standards is preferably determined based on the amount of the first tag or the second tag, preferably the first tag, which is attached to said internal peptide standard or internal peptide standards. In an embodiment thereof, the tag comprises a fluorescence label known to a person skilled in the art and of which some non-limiting examples are disclosed herein. The amount of the tag is determined by measuring the fluorescence signal of the label. In an alternative embodiment, the tag comprises a UV label known to a person skilled in the art and of which some non-limiting examples are disclosed herein. The amount of the tag is determined by measuring the UV signal of the label.

In each and any of the first, second and third basic embodiment of the method of the invention for determining the presence and/or quantity of a target polypeptide of which a first peptide is a proteotypic peptide, in at least one mixture of different polypeptides, preferably the mixture of different polypeptides is preferably a sample. In an embodiment such sample contains at least one polypeptide, preferably more than one polypeptide. The sample may be a biological sample or a non-biological sample. A biological sample may be a sample obtained from biological material. The biological material may be selected from the group comprising a body fluid sample, a lysate or preparation of a cell or a part thereof, a lysate or preparation of a tissue or a part thereof, a lysate or preparation of an organ or part thereof, a lysate or preparation of a complete organism or part thereof, a fermentation broth or part thereof and a cell culture fluid or part thereof The body fluid sample may be a sample of a body fluid, whereby the body fluid is selected from the group comprising blood, plasma, serum, urine, liquor, sputum, faeces The non-biological sample may be sample from a chemical reaction. The chemical reaction may be a synthesis of polypeptides or a process of chemical modification of polypeptides simulating posttranslational modifications.

To the extent it is indicated in the methods of the invention that the mixture of different polypeptides preferably contains a or the target polypeptide, preferably, this is to mean that the mixture either contains said target polypeptide or does not contain said target polypeptide. In a preferred embodiment, the mixture contains said target polypeptide. It is obvious to a person skilled in the art that if the mixture does not contain said target polypeptide, its amount cannot be determined. Nevertheless, the finding when performing the method of the invention for determining the presence and/or quantity of a target polypeptide of which a first peptide is a proteotypic peptide, in at least one mixture of different polypeptides, that the mixture does not contain said target polypeptide, may already constitute a valuable piece of information and a valuable result obtained by said method.

To the extent it is indicated in the methods of the invention that the amount of the internal peptide standard of the invention or of the panel of internal peptide standards of the invention which is added to a compound, vessel reaction mixture or something similar, is unknown, preferably, this is to mean that the number of molecules of the internal peptide standard of the invention or of the panel of internal peptide standards of the invention is unknown which is added to said compound, vessel, reaction mixture or something similar. In connection therewith it is to be acknowledged that the adding of the internal peptide standard of the invention or of the panel of internal peptide standards of the invention preferably occurs by transferring an aliquot of a liquid containing said internal peptide standard of the invention or of the panel of internal peptide standards of the invention. Preferably the volume of the aliquot is known in the performing of the method of the invention. However, it is also within the present invention that the volume of the aliquot is not known. Particularly in the latter embodiment, however, it is preferred that the volume of the reaction mixture which is obtained by adding said internal peptide standard of the invention or of the panel of internal peptide standards of the invention to said compound, vessel, reaction mixture or something similar, is known.

As preferably used, any wording which specifies the limits of a range such as, e. g., “from 1 to 5” means any integer from 1 to 5, i. e. 1, 2, 3, 4 and 5. In other words, any range that is defined by two integers comprises both the two integers defining said limits of the definition and any integer comprised by or contained within said range.

Further features, embodiments and advantages of the present invention may be taken from the following figures and examples, whereby

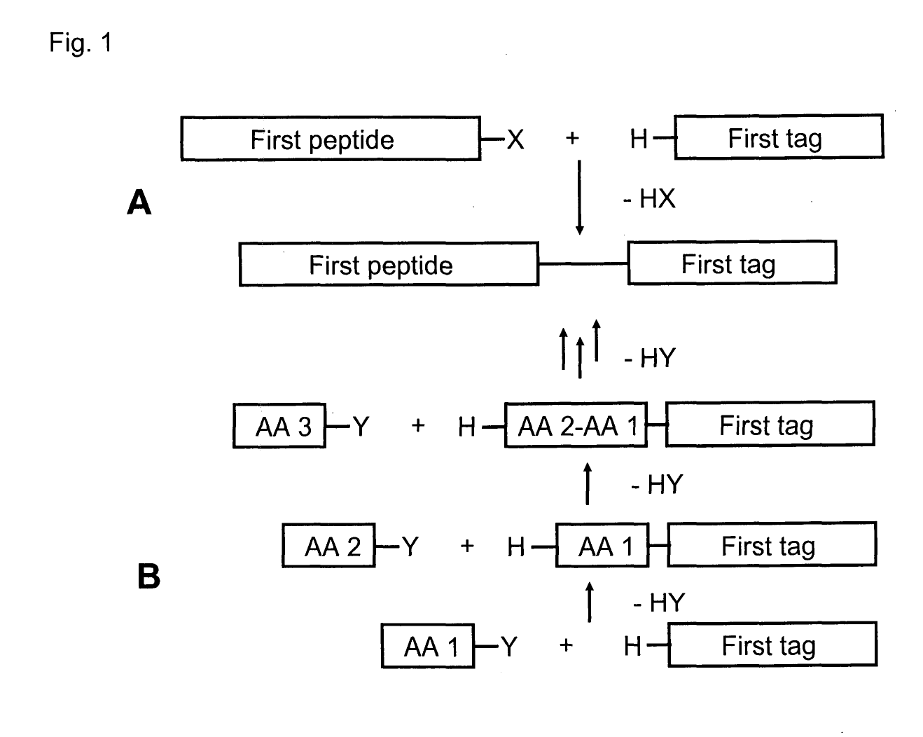

FIG. 1 is a schematic representation illustrating the generation of an internal peptide standard of the invention;

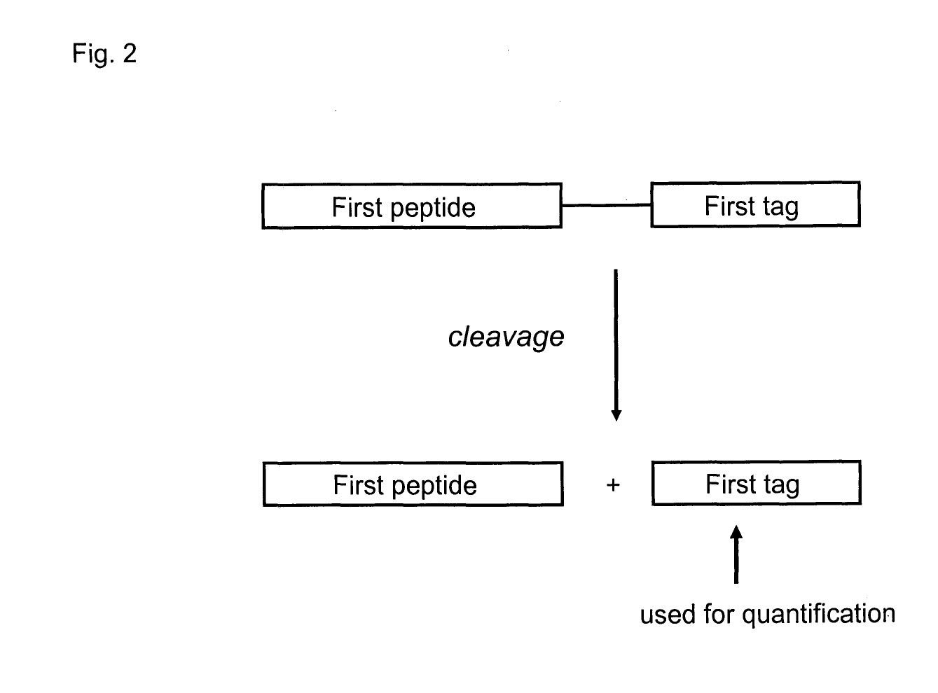

FIG. 2 is a schematic representation illustrating the quantification of a purified internal peptide standard of the invention;

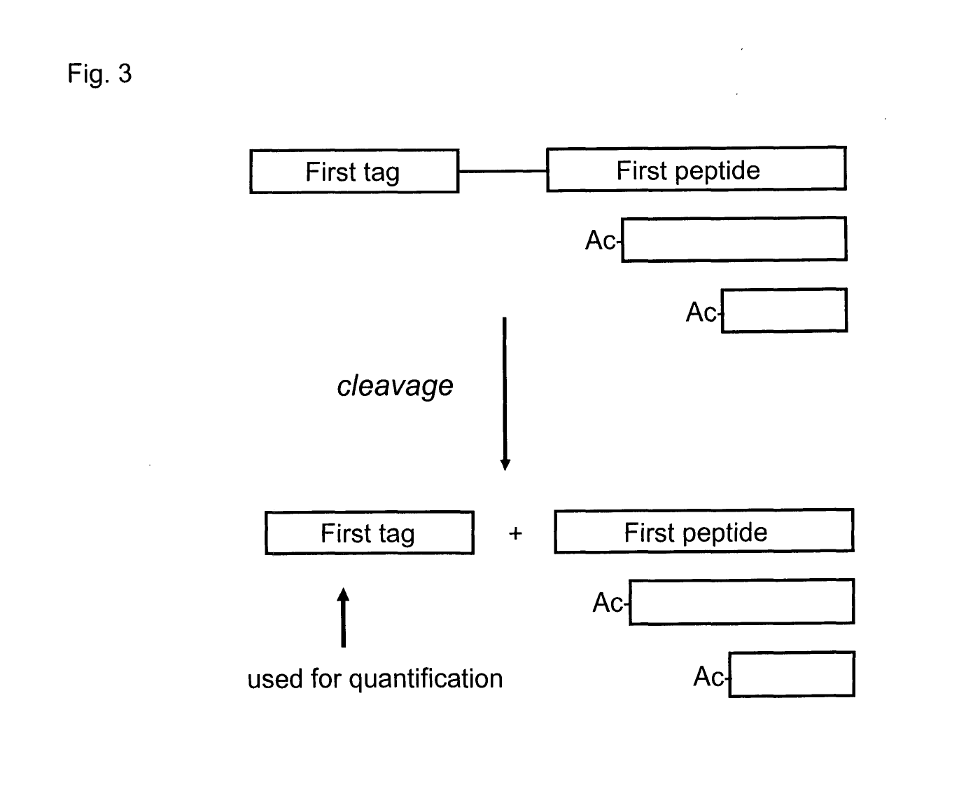

FIG. 3 is a schematic representation illustrating the quantification of a non-purified internal peptide standard of the invention which is synthesized with capping steps;

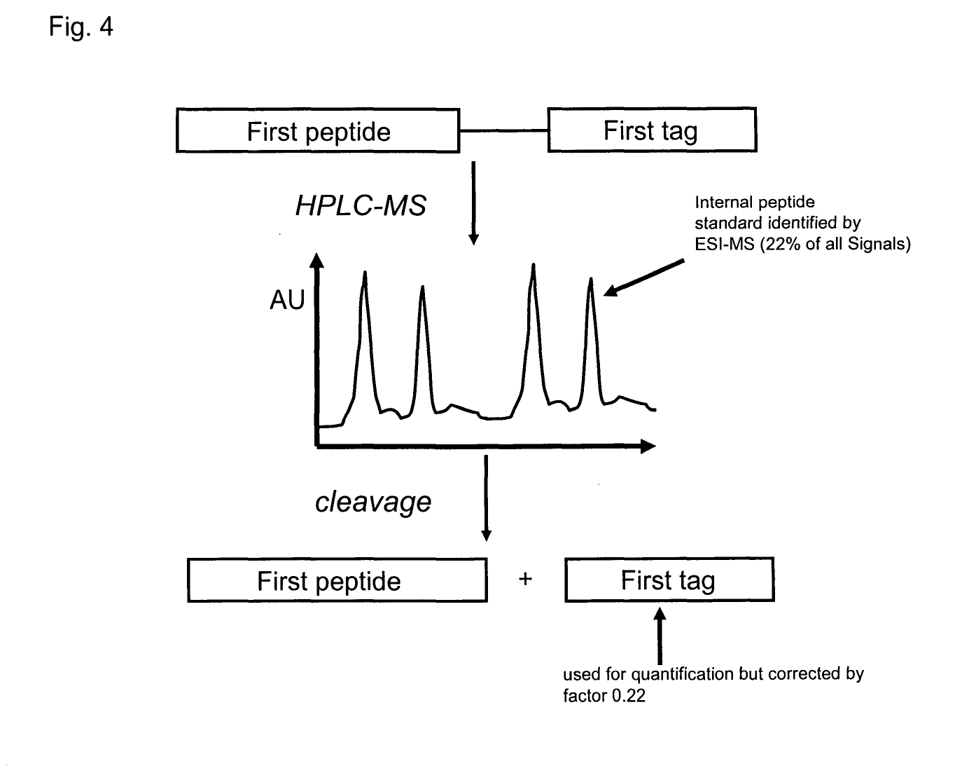

FIG. 4 is a schematic representation illustrating the quantification of a non-purified internal peptide standard analysed by HPLC-MS;

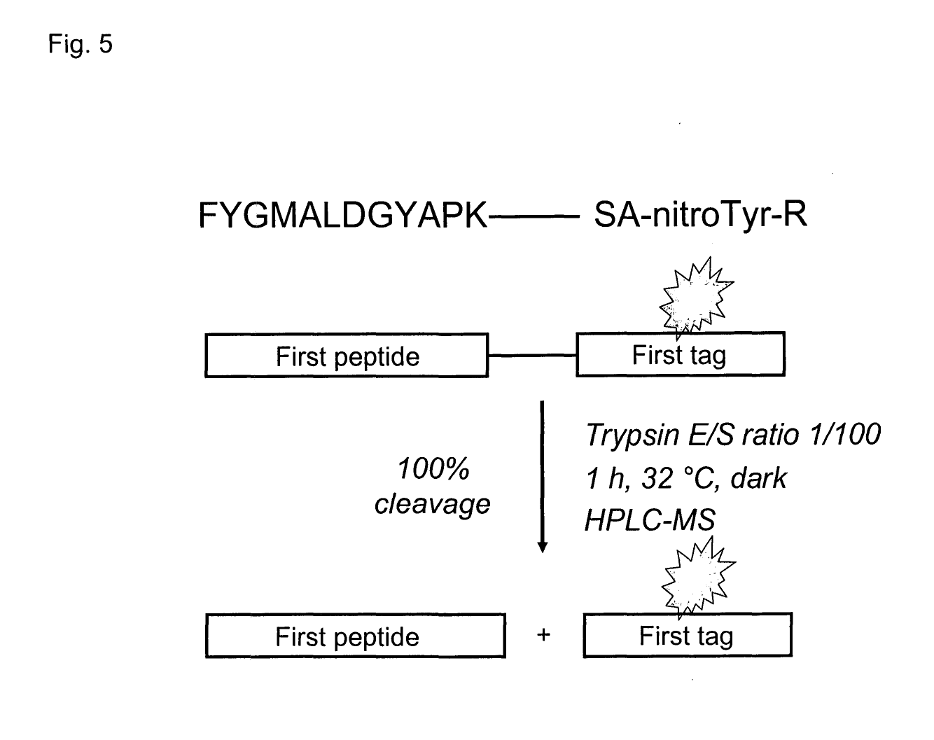

FIG. 5 is a schematic representation illustrating the experiment showing the compatibility of chromophore incorporated into a first tag with enzymatic cleavage of the internal peptide standard, where FYGMALDGYAPK-SA-nitroTyr-R corresponds to SEQ ID NO:71;

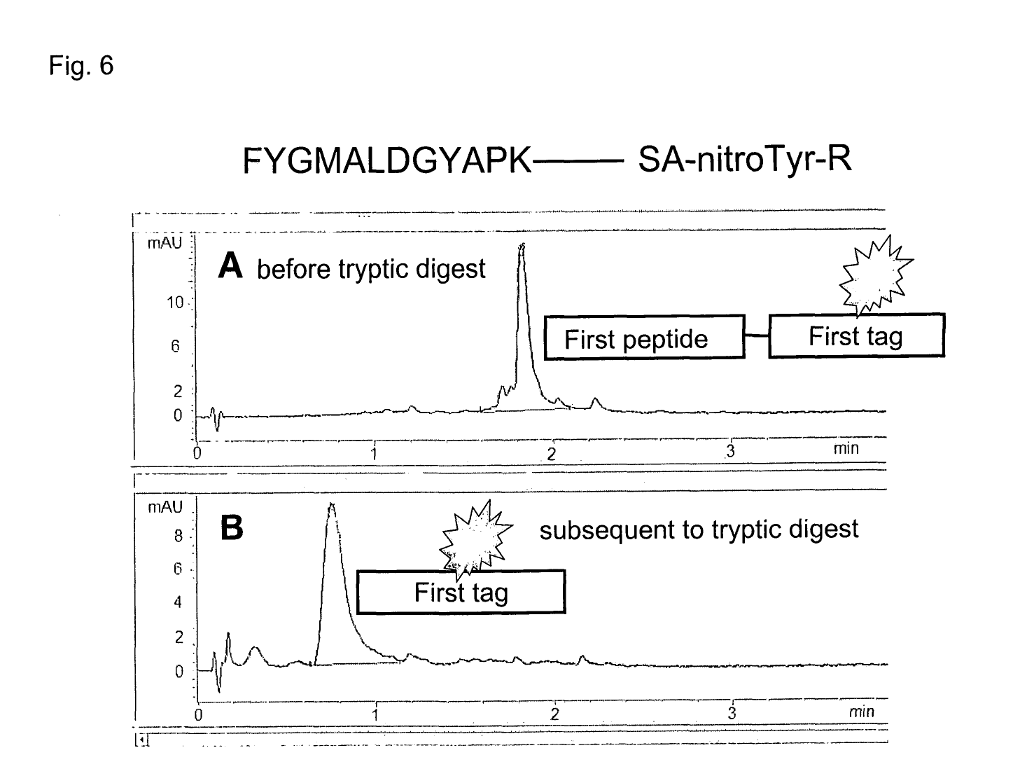

FIG. 6 is a chromatogram indicating the run characteristics of a first peptide labelled with a first tag prior to and subsequent to tryptic digest as discussed in Example 2, where FYGMALDGYAPK-SA-nitroTyr-R corresponds to SEQ ID NO:71;

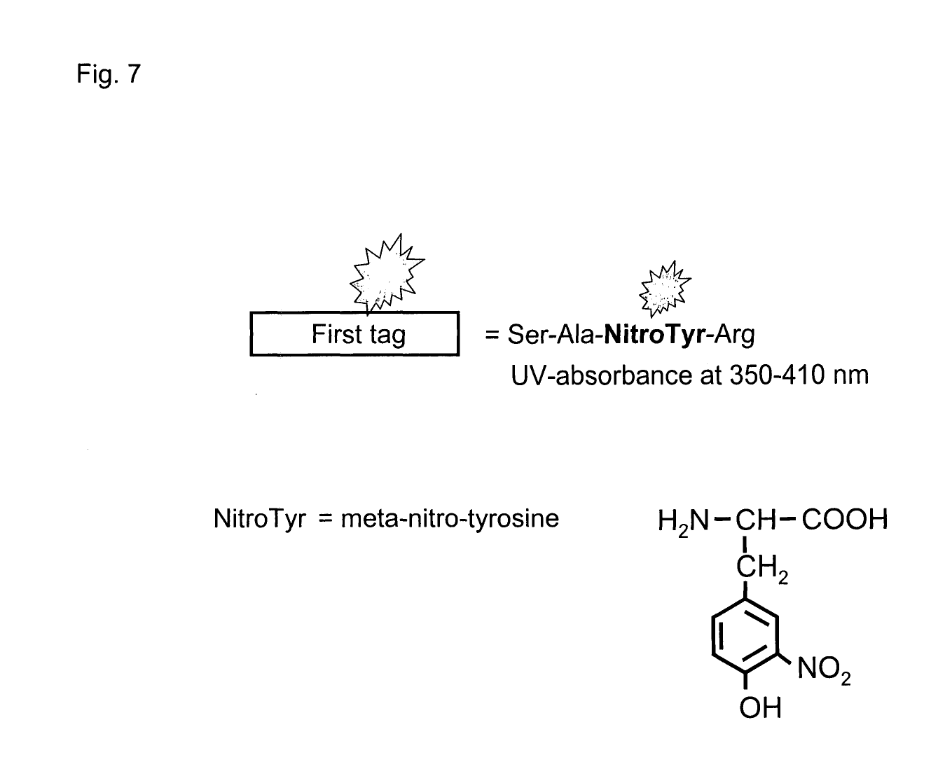

FIG. 7 shows the composition of a first tag containing meta-nitro-tyrosine, where Ser-Ala-NitroTyr-Arg corresponds to SEQ ID NO:1;

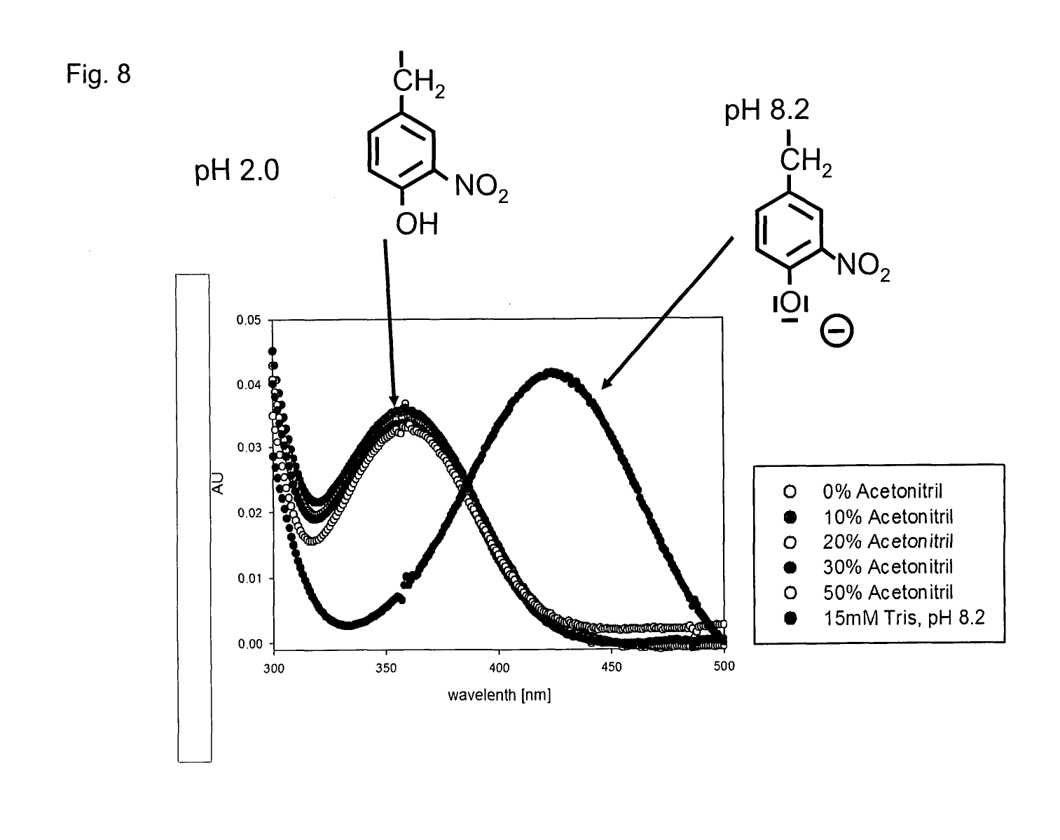

FIG. 8 shows UV-Vis spectra of acetylated first tag containing meta-nitro-tyrosine as discussed in Example 3;

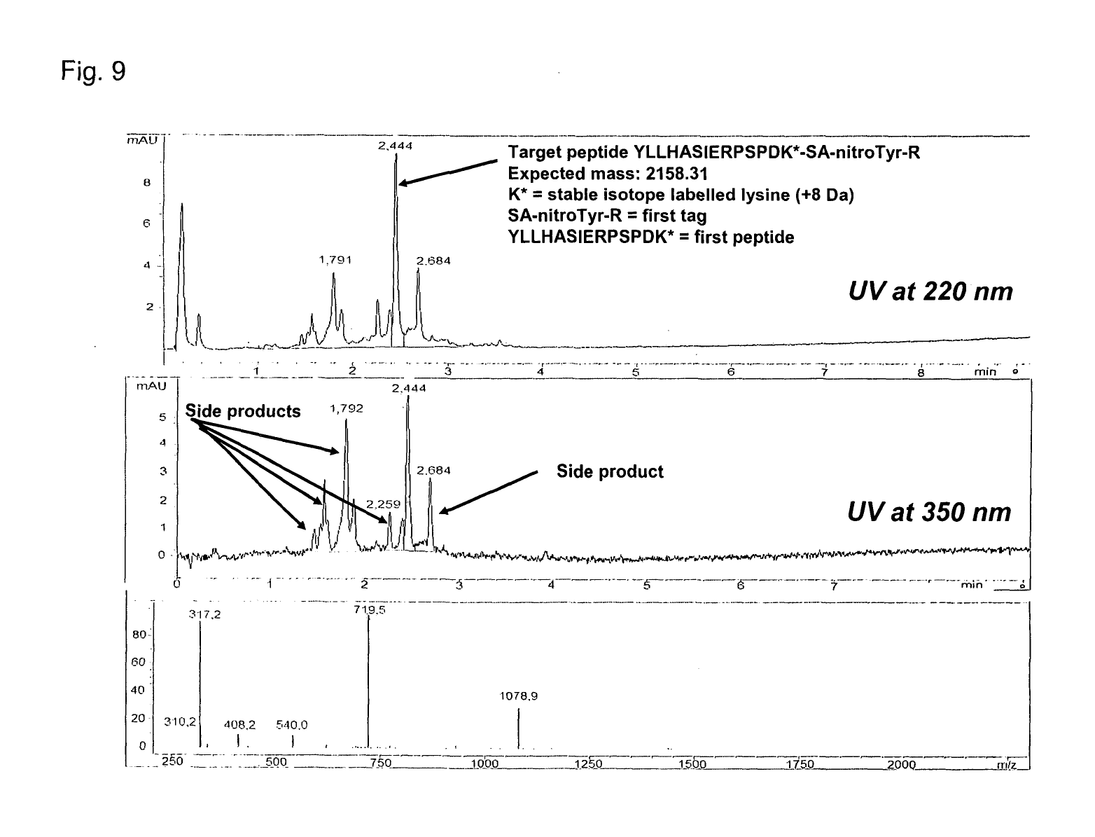

FIG. 9 shows the result of a quantification of non-purified internal peptide standard analysed by HPLC-ESI-MS equipped with a UV-Vis detector, where YLLHASIERPSPDK*-SA-nitroTyr-R corresponds to SEQ ID NO:72, SA-nitroTyr-R corresponds to SEQ ID NO:1, and YLLHASIERPSPDK* corresponds to SEQ ID NO:73;

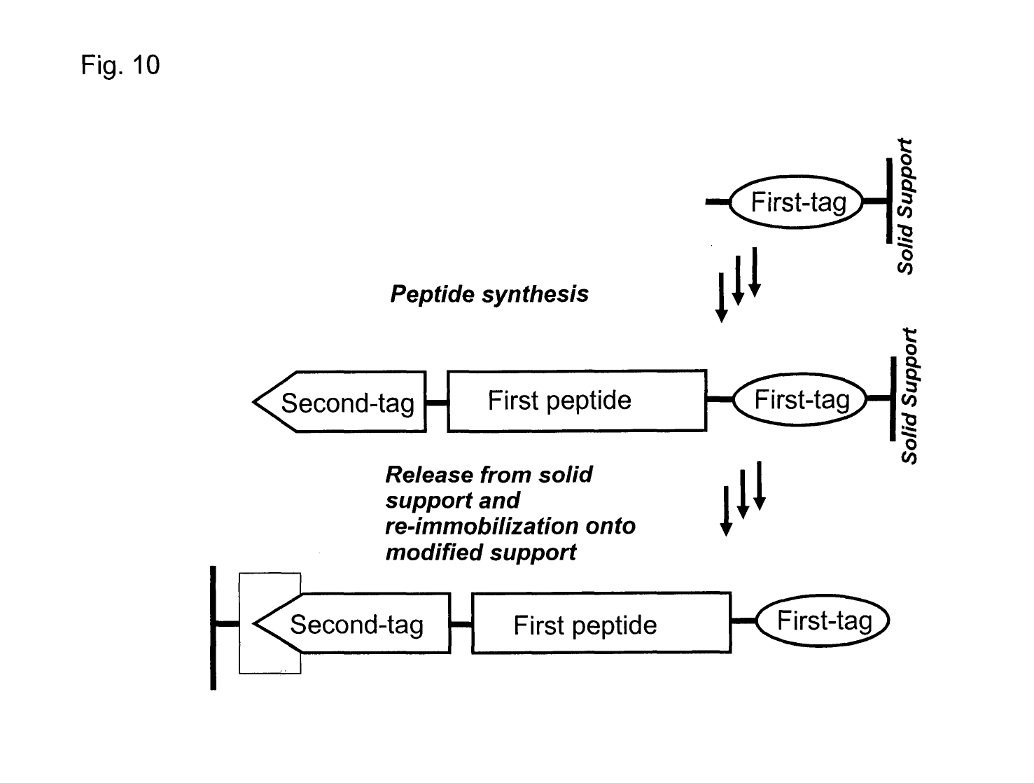

FIG. 10 is a schematic representation illustrating the generation and use of an internal peptide standard comprising both a first and a second tag;

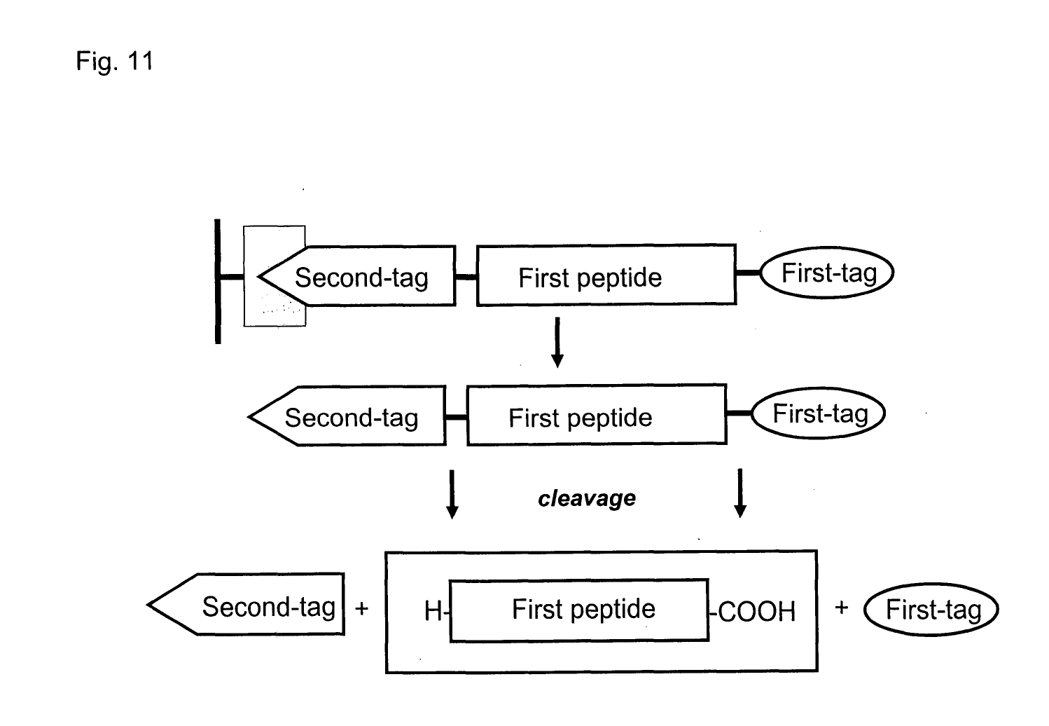

FIG. 11 is a schematic representation illustrating off-support cleavage of internal peptide standard;

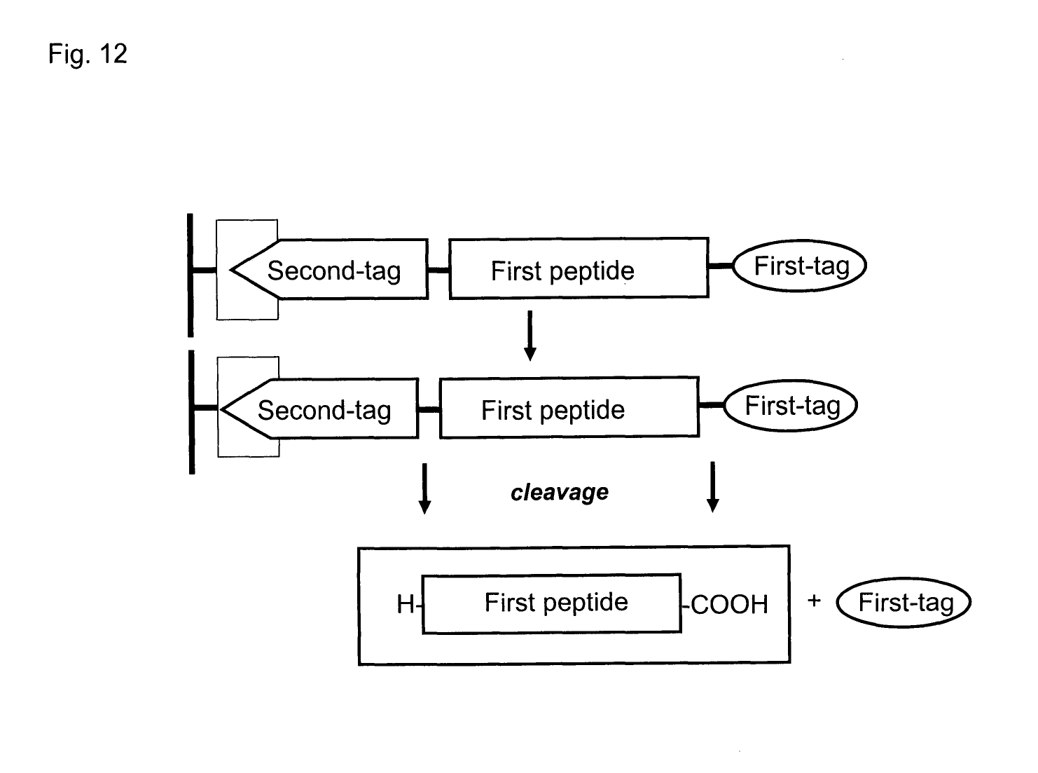

FIG. 12 is a schematic representation illustrating on-support cleavage of internal peptide standard;

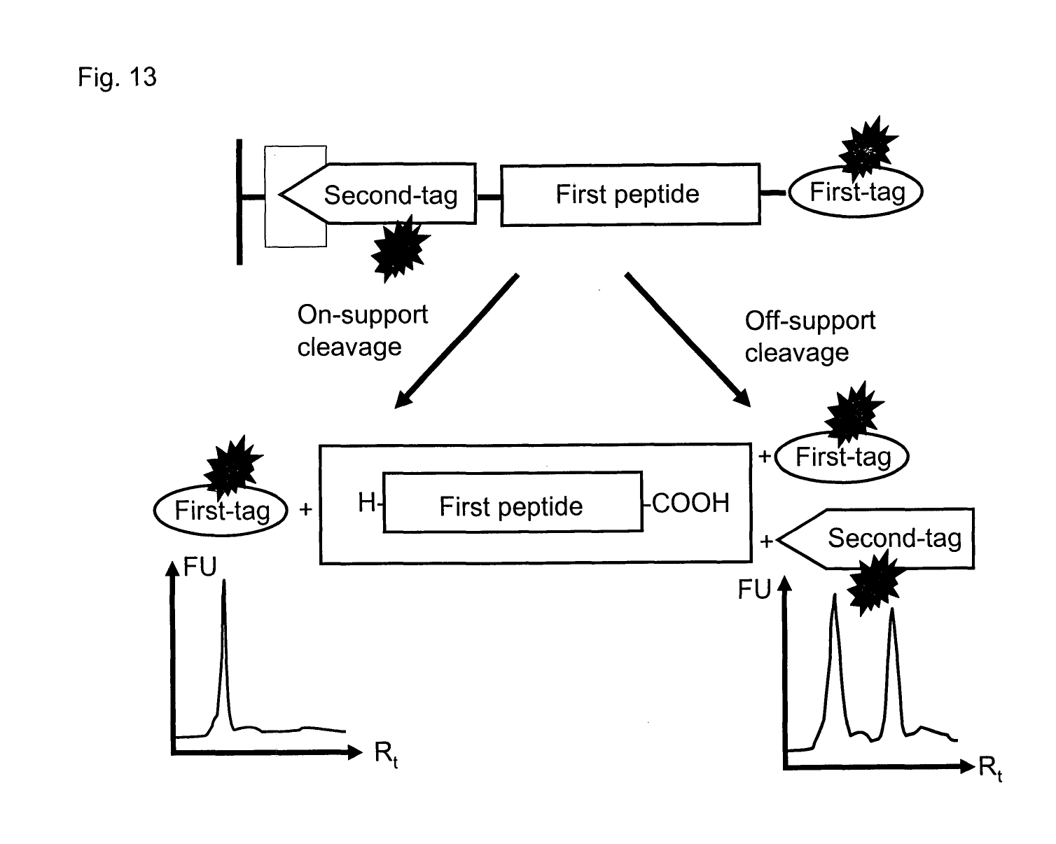

FIG. 13 is a schematic representation illustrating various options for quantifying first peptides released by cleavage of purified internal peptide standards;

FIG. 14 shows the composition of a second tag containing desthio-biotin;

FIG. 15 shows the composition of first and second tag containing fluorescence label;

FIGS. 16 and 17 indicate various chemoselective chemical reactions which can be used for immobilization of peptide derivatives onto supports; and

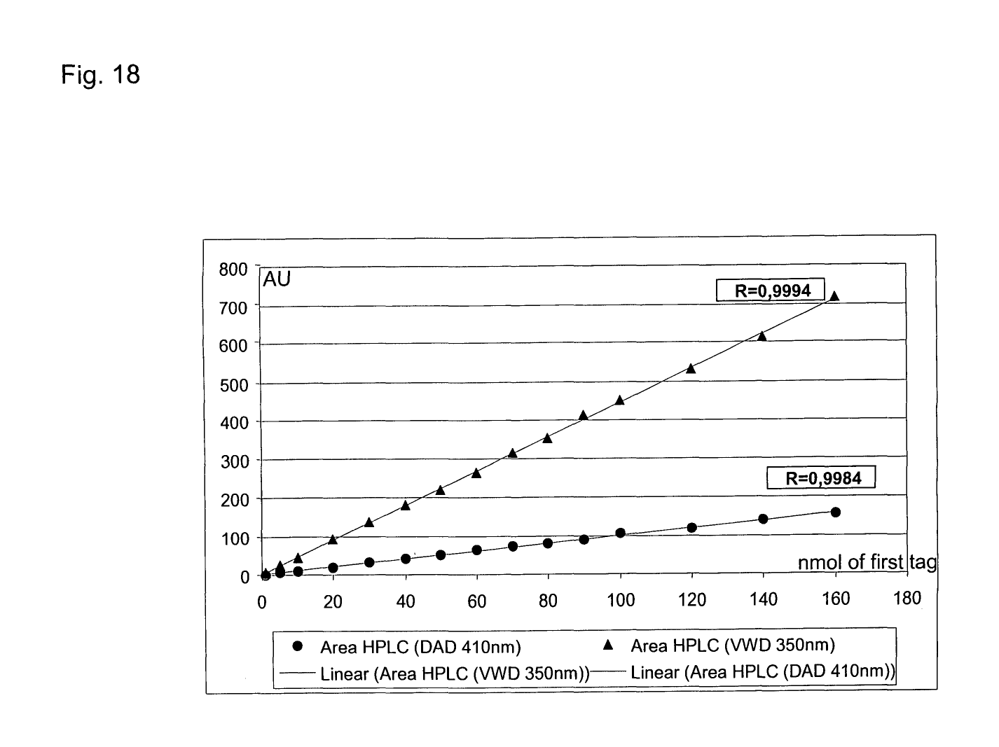

FIG. 18 is a diagram indicating absorption units as a function of the concentration of the first tag illustrated in FIG. 7 whereby absorption is determined at either 350 nm or 410 nm.

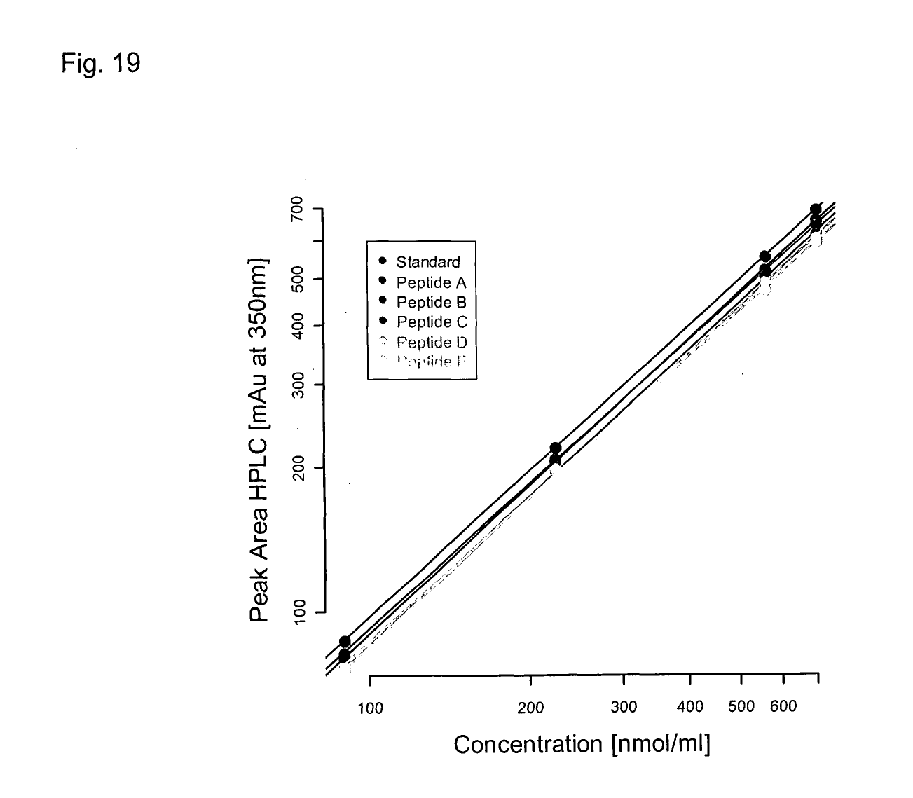

FIG. 19 shows straight calibration lines for the quantification of five different labeled peptides and the standard Ac-SA-nitroTyr-R-OH (SEQ ID NO:1).

FIG. 1 is a schematic representation for the generation of an internal peptide standard of the invention. In FIG. 1 A) a) pre-synthesized first tag is fused to the pre-synthesized first peptide in one step yielding the internal peptide standard. As illustrated in FIG. 1 B) if the first tag is synthesized, in a step by step manner, additional amino acids are added in a stepwise manner according to peptide synthesis protocols of the state of the art such as described in EP 0 651 762. After the coupling of the last amino acid residue the amino acid sequence of the first peptide is completed and subsequent to removal of protecting groups and optional removal of the compound from a solid synthesis support the synthesis of the internal peptide standard is completed.

FIG. 2 is a schematic representation illustrating the quantification of a purified internal peptide standard of the invention. The purified internal peptide standard, ideally 100% pure or at least 98% pure or 95% pure, is cleaved with a means for sequence-specific cleavage to release the first tag and the first peptide in a defined ratio. Preferably, a ratio is 1:1. If the cleavage reaction is nearly 100% and if the starting purity is high the amount of the released first tag is proportional to the amount of the released first peptide.

FIG. 3 is a schematic representation illustrating the quantification of a non-purified internal peptide standard of the invention which is synthesized with capping steps. In this embodiment, the internal peptide standard is synthesized by a modified procedure. After each coupling step and before each removal of the protecting group there is an additional capping step introduced. If, e.g., appropriately activated acetic acid is used, the side products resulting from incomplete coupling steps are acetylated and thereby no longer chemically reactive. At the end of the synthesis of the first peptide the first tag is added. Because of the capping steps the first tag will react with the first peptide only and not with acetylated impurities. At the end of the synthesis the desired internal peptide standard is created together with acetylated impurities which do not carry the first tag. Subsequent to a cleavage reaction the first tag could be released from the first peptide only. Therefore, in this case the amount of released first tag is indicative for the amount of release first peptide.

FIG. 4 is a schematic representation illustrating the quantification of a non-purified internal peptide standard analysed by HPLC-MS. The internal peptide standard is synthesized and analysed by HPLC coupled to a mass spectrometer equipped with an UV-Vis detector. During the HPLC run the internal peptide standard is separated from the impurities which still carry the first tag. The signal for the internal peptide standard can be identified by the mass spectrometer coupled to the HPLC. The relative amount of first tag bound to the first peptide could be calculated from the UV-Vis trace of the HPLC run. This calculation yields the relative amount of internal standard in percent within the non-purified internal peptide standard. Subsequent to an appropriate cleavage reaction the amount of the released first tag is measured and then corrected for the relative content of the starting material. In this example the UV-Vis trace yielded 22% of all first tag containing peptides which are corresponding to the internal peptide standard which was identified by the mass trace. Subsequent to cleavage the measured total amount of released first tag has to be corrected by a factor of 0.22 reflecting the 22% content of the internal peptide standard.

FIG. 7 indicated the composition of a first tag containing meta-nitro-tyrosine. The composition of a first tag is shown using the one letter code for the naturally occurring amino acid residues. This compound is a representative example of a first tag optimized for cleavage using protease trypsin. Serine and alanine residues are occupying the P1′ and P2′-position, respectively. The chromophor-containing amino acid residue meta-nitro-tyrosine is optimized for the P3′-position. In the P4′-position an arginine residue is used. The structure of the modified amino acid residue meta-nitro-tyrosine is given. Depending on the protonation state of the hydroxyl group of this residue the aromatic ring systems have a strong absorbance with a maximum at 350 nm for the protonated state of the hydroxyl and a maximum at 410 nm for the dissociated form of the hydroxyl group.

FIG. 10 is a schematic representation illustrating the generation and use of an internal peptide standard comprising both a first and a second tag. More complex peptide standards can be produced using peptide synthesis as illustrated in FIG. 10. Subsequent to generation of the first tag on a solid support, either by stepwise synthesis of by attachment of a pre-synthesized tag directly to the solid support, a first peptide followed by a second tag are synthesized in a stepwise manner. Alternatively, the first peptide is attached to the first tag and then the second tag is attached to the bound first-peptide-first-tag structure. Additionally, combinations of stepwise synthesis and attachment of pre-synthesized structures are possible.

FIG. 11 is a schematic representation illustrating off-support cleavage of internal peptide standard comprising both a first tag and a second tag. Internal peptide standard immobilized to a support via the second tag could be release from that support subsequent to washing steps. These washing steps will remove side products of peptide synthesis which did not bound specifically or which did not formed a covalent bond. The released, and purified, internal peptide standard could be cleaved yielding the first peptide together with the second and the first tag. The first tag and/or the second tag can be used for quantification of the amount of the first peptide generated by the cleavage reaction.

FIG. 12 is a schematic representation illustrating on-support cleavage of internal peptide standard comprising both a first tag and a second tag. Internal peptide standard immobilized to a support via the second tag can be cleaved directly on the support subsequent to washing steps. These washing steps will remove side products of peptide synthesis which did not bound specifically or which did not formed a covalent bond. The purified but still immobilized internal peptide standard can be cleaved on the support yielding the first peptide together with the first tag. The second tag is still bound to the support (not shown in the figure). The first tag could be used for quantification of the amount of the first peptide generated by the cleavage reaction.

FIG. 14 shows the composition of a second tag containing desthio-biotin. The composition of a second tag is shown using the three-letter-code for the naturally occurring amino acid residues. This illustrated second tag is an embodiment of a second tag optimized for cleavage using protease trypsin. Arginine residue is occupying the P1-position. The proline residue is known to be preferred by the protease trypsin in the P2-position. The alanine residue is well accepted in the P3-position. Desthio-biotin is used as a reactive moiety within the second tag enabling selective binding to supports modified with biotin/desthiobiotin binding proteins like avidin or streptavidin. To space apart the desthio-biotin residue from the amino acid residues and to allow effective interaction of the desthio-biotin with the binding pockets on the desthio-biotin binding support a linker molecule, Ttds, was introduced. The chemical structure of the linker is shown together with the chemical structure of desthio-biotin. The desthio-biotin is used instead of biotin because the affinity of desthio-biotin to the proteins streptavidin or avidin is much lower than the affinity of biotin. This enables elution of bound, desthio-biotin-containing internal peptide standards from the supports using biotin as a reagent effectively competing with the desthio-biotin-modified compounds.

FIG. 15 shows the composition of first and second tag containing fluorescence label. The composition of a first tag and second tag is shown using the three-letter-code for the naturally occurring amino acid residues. The indicated first tag and second tag each represents a first and second tag optimized for cleavage using protease trypsin. In the first tag serine and alanine residues are occupying the P1′ and P2′-position, respectively. The fluorophore-containing amino acid residue Cal=β-7-methoxy-Coumaryl-alanine is optimized for the P3′-position. In the P4′-position a glycine residue is used. In the second tag an arginine residue is occupying the P1-position. The proline residue is known to be preferred by the protease trypsin in the P2-position. The Cal=β-7-methoxy-Coumaryl-alanine residue is well accepted in the P3-position in the case of trypsin as cleavage-mediating protease. The substituted coumaryl-residue in that alanine derivative yielded a highly fluorescent signal. Desthio-biotin is used as a reactive moiety within the second tag enabling selective binding to supports modified with biotin/desthiobiotin binding proteins like avidin or streptavidin. To space apart the desthio-biotin residue from the amino acid residues and to allow effective interaction of the desthio-biotin with the binding pockets on the desthio-biotin binding support a linker molecule, Ttds, was introduced. The chemical structure of the linker together with the chemical structure of desthio-biotin is shown in FIG. 14. The desthio-biotin is used instead of biotin because the affinity of desthio-biotin to the proteins streptavidin or avidin is much lower than the affinity of biotin. This enables elution of bound, desthio-biotin-containing internal peptide standards from the supports using biotin as a reagent effectively competing with the desthio-biotin-modified compounds.

FIG. 16 indicates various chemoselective chemical reactions which can be used for immobilization of peptide derivatives onto supports. Four different chemical reactions are shown which are suitable for chemoselective formation of a covalent bond between an internal peptide standard and an appropriately modified support. In reaction A) support bound aldehydes (R₁=hydrogen) or ketones (R₁ not hydrogen) and amino-oxy-moiety containing internal peptide standards react by formation of oxime-bonds. In reaction B) support bound mercaptanes and maleinimide containing internal peptide standards react by formation of substituted succinimides. In reaction C) support bound hydrazides and aldehyde containing internal peptide standards react by formation of substituted hydrazones. In reaction D) support bound quinines and cyclopentadiene containing internal peptide standards react by formation of substituted Diels-Alder-Adducts.

FIG. 17 indicates further chemoselective chemical reactions which can be used for immobilization of peptide derivatives onto supports. Four different chemical reactions are shown which are suitable for chemoselective formation of a covalent bond between an internal peptide standard and an appropriately modified support. In reaction A) support bound aldehydes (R=hydrogen) or ketones (R not hydrogen) and substituted hydroxyl-amines within the internal peptide standards react by formation of nitrones. In reaction B) support bound aldehydes (R=hydrogen) and aromatic amino carboxyl containing internal peptide standards react by formation of substituted 2,3,4a,8a-tetrahydro-1H-quinazoline-4-ones. In reaction C) support bound thioamides and alpha-bromo-ketone containing internal peptide standards react by formation of substituted thiazoles. In reaction D) support bound thioamides and substituted phenylene diamine containing internal peptide standards react by formation of substituted benzoimidazoles.

FIG. 19 shows straight calibration lines for the quantification of five different labeled peptides. All peptides carry the C-terminal quantification-tag-SA-nitroTyr-G-OH (SEQ ID NO:2, Peptide A-E, Table 1) that allows quantification by UV measurement at 350 nm. Also the calibration line for the external standard Ac-SA-nitroTyr-R-OH (SEQ ID NO:1) is depicted.

### EXAMPLE 1

### Compatibility of Chromophore Incorporated into First Tag with Enzymatic Cleavage of the Internal Peptide Standard

The basic experiment subject to this example is illustrated in FIG. 5.

The internal standard peptide of the given sequence indicated as the one letter code for the naturally occurring amino acid residues, was synthesized in a stepwise manner on a solid support. The first tag contains a modified amino acid residue (nitroTyr=3′-nitro-tyrosine) which yielded a specific UV-Vis absorption at wavelengths between 350-410 nm. The peptide was released from the solid support and treated with the protease trypsin for 1 hour at 32° C. Before and subsequent to the cleavage reaction a HPLC-MS analysis was performed to estimate the effectiveness of cleavage reaction. The basic experiment is illustrated in FIG. 5.

From this example it was concluded that the incorporation of a chromophore into a first tag is compatible with enzymatic cleavage of the internal peptide standard.

### EXAMPLE 2

### UV-Vis Traces at 410 nm Before and Subsequent to Cleavage of Internal Peptide Standard

The internal peptide standard depicted in FIG. 7 was analysed using HPLC using the one letter code for the naturally occurring amino acid residues. The UV-Vis traces at 410 nm are shown. FIG. 6 A) indicates the UV-trace of reaction solution before addition of the protease trypsin. The internal peptide standard containing the meta-nitro-tyrosine absorbing at 410 nm, elutes from the PR-18 column with a retention time of about 1.85 minutes. FIG. 6 B) indicates the UV-trace of reaction solution subsequent to addition of the protease trypsin and incubation of the reaction mixture at 32° C. for one hour. The released first tag containing the meta-nitro-tyrosine absorbing at 410 nm, elutes from the PR-18 column with a retention time of about 0.8 minutes. The released first peptide is not visible at that wavelength. Because there is nearly no remaining signal visible at retention time of about 1.85 minutes the cleavage reaction seem to be very effective with a cleavage rate close to 100%.

The result of the experiment underlying this example is illustrated in FIG. 6.

This result demonstrates, that the meta-nitro-tyrosine residue in the first tag is compatible with cleavage reactions mediated by protease trypsin.

### EXAMPLE 3

### UV-Vis Spectra of Acetylated First Tag Containing Meta-Nitro-Tyrosine

The UV-Vis spectra in the range of 300-500 nm are shown in FIG. 8 for acetylated first tag Ser-Ala-mNitroTyr-Arg (SEQ ID NO:1). The spectra were recorded for solution of tag in different acetonitrile/water mixtures containing trifluoro acetic acid. Caused by the trifluoro acetic acid the pH value of these mixtures is in the range of pH 2. Additionally, a UV-Vis spectrum of the acetylated tag Ser-Ala-mNitroTyr-Arg (SEQ ID NO:1) dissolved in 15 mM TRIS buffer pH 8.2 was recorded. The maximum of absorbance in the measured range is about 420 nm for pH 8.2 but about 355 nm for pH 2.0. The reason for that difference is the protonation state of the side chain of the meta-nitro-tyrosine residue. The different structures for the different protonation states are shown. On the left hand side the structure for the protonated meta-nitro-tyrosine residue is shown. This is the predominant moiety at pH 2.0. On the right hand side the structure for the dissociated meta-nitro-tyrosine residue is shown. This is the predominant moiety at pH 8.2 caused by the increased acidic character of the phenolic group in proximity to the nitro function.

The very similar UV-Vis spectra at pH 2.0 for the different solvent mixtures enable quantification of first tags containing the meta-nitro-tyrosine using HPLC gradients. The total absorbance at 355 nm is nearly independent on the acetonitrile content of the solvent.

### EXAMPLE 4

### Quantification of Non-Purified Internal Peptide Standard Analysed by HPLC-MS

The result of this quantification is shown in FIG. 9

The internal peptide standard is analysed by HPLC-ESI-MS equipped with a UV-Vis detector. On top of FIG. 9 the UV trace at 220 nm is shown. There are several signals representing different impurities besides the target internal peptide standard which could be identified in the ESI mass spectrum within the retention time region of 2.425-2.502 minutes. In the middle of FIG. 9 the UV-trace at 350 nm is shown which is indicative for the first tag which in this case is equipped with a chromophor (meta-nitro-tyrosine) absorbing specifically at 350 nm at pH value of 2. Comparing the signals in the UV-Vis trace at 220 nm with the signals in the UV-Vis trace at 350 nm it becomes clear that nearly all of the side products having the first tag. Nevertheless, the areas of the different peaks at 350 nm allow the exact quantification of each side product and of the target internal standard peptide if the HPLC_MS system was calibrated with a defined amount of meta-nitro-tyrosine-containing first tag.

### EXAMPLE 5

### Generation and Use of an Internal Peptide Standard Comprising Both a First and a Second Tag

More complex peptide standards can be produced using peptide synthesis as illustrated in FIG. 10. Subsequent to generation of the first tag on a solid support, either by stepwise synthesis of by attachment of a pre-synthesized tag directly to the solid support, a first peptide followed by a second tag are synthesized in a stepwise manner. Alternatively, the first peptide is attached to the first tag and then the second tag is attached to the bound first-peptide-first-tag structure. Additionally, combinations of stepwise synthesis and attachment of pre-synthesized structures are possible.

At releasing the internal peptide standard by appropriate methods from the solid support the internal peptide standard can be re-immobilized onto another support via moieties within the second tag. This immobilization reaction can be selected from a chemoselective formation of a chemical bond or from the specific binding of the second tag or part of the second tag to binding partners or interaction partner.

### EXAMPLE 6

### Methods for Quantifying First Peptides Released by Cleavage of Purified Internal Peptide Standards

As illustrated in FIG. 13, an internal peptide standard comprising both a first tag and a second tag, is immobilized to a support. Both the second and the first tag are labeled with a fluorescent amino acid residue. Subsequent to immobilization of the non-purified internal peptide standard the support is washed to remove acetylated side products of peptide synthesis which did not bound specifically or which did not formed a covalent bond. Subsequently, the purified internal peptide standard can be either cleaved directly on the support (left hand side of FIG. 13) or after release from the support (off-support cleavage, right hand side of FIG. 13). After on-support cleavage the second tag will remain on the support and the first peptide together with the first tag will be release from the support. The released mixture of first peptide and first tag can be analyzed by HPLC equipped with a fluorescence detector. If the system is calibrated with fluorescent reference first tag, the fluorescence intensity (measured in fluorescence units, FU) of the released first tag can be used for quantification of the first tag and therefore for the quantification of the first peptide.

After off-support cleavage the first peptide together with the first tag and the second tag will be release from the support. The released mixture of first peptide, first tag and second tag can be analyzed by HPLC equipped with a fluorescence detector. If the system was calibrated with fluorescent reference first tag and or fluorescent second tag, the fluorescence intensities, measured in fluorescence units, FU, of the released first tag and second tag could be used for quantification of the tags and therefore for the quantification of the first peptide.

### EXAMPLE 7

### Calibration of HPLC System Using Defined Amounts of Chromophor-Containing First Tag

The acetylated form of a first tag (Ac-Ser-Ala-NitroTyr-Arg (SEQ ID NO:1), see FIG. 7) was synthesized and absolutely quantified by amino acid analysis. Defined amount of quantified, acetylated first tag were loaded onto the HPLC column and the resulting areas—absorption units, AU—of the peaks detected at either 350 nm or 410 nm were plotted against total amount of acetylated first tag. Linear regression analysis was performed for the two series of experiments yielding correlations of 0.9994 and 0.9986, respectively. The result is indicated in FIG. 18.

The linear correlation which is evident form FIG. 18 enables determination of amounts of meta-nitro-tyrosine-containing first tag released from appropriate internal peptide standards. As is also evident from FIG. 18, determining absorption at 350 nm is clearly advantageous compared to absorption at 410 nm in terms of the slope observed which allows a more accurate determination of the first tag.

### EXAMPLE 8

### Peptide Quantifications with a Nitro-Tyrosine-Based Tag: Linearity and Independence from Peptide Sequence

Five different peptides carrying the C-terminal quantification-tag-SA-nitroTyr-G-OH ((SEQ ID NO:2), Peptide A-E, Table 1) were synthesized by Fmoc-based solid phase peptide synthesis and purified by HPLC. FIG. 19 shows linear calibration curves for the quantification of the five peptides via HPLC in comparison to the external standard Ac-SA-nitroTyr-R-OH (SEQ ID NO:1). Quantification was performed at 350 nm.

It turned out that the quantification was linear over the tested concentration range (100-700 nmol/ml) for all peptides and independent of the peptide sequence. The overall error (SD) for quantification was 5.4%.

TABLE 1
Peptides quantified by UV absorption of a nitro-
Tyrosine-based quantification tag.

| | | SEQ |
|---|---|---|
| | | ID |
| Name | Sequence | NO: |
| Standard | Ac-SA-nitroTyr-R-OH | 1 |
| Peptide A | H-LTGEVVALK-SA-nitroTyr-G-OH | 3 |
| Peptide B | H-LIDWGLAEFYHPAQEYNV-R-SA-nitroTyr- | 4 |
| | G-OH | |
| Peptide C | H-EQSQPCADNAVLSSGLTAA-R-SA-nitroTyr- | 5 |
| | G-OH | |
| Peptide D | H-ELIFQETA-R-SA-nitroTyr-G-OH | 6 |
| Peptide E | H-LAYTFEVQK-SA-nitroTyr-G-OH | 7 |

### EXAMPLE 9

### Peptide Quantifications with a Nitro-Tyrosine-Based Tag: Efficiency of Tag Cleavage from 23 Different Peptides

23 peptides carrying the C-terminal quantification-tag-SA-nitroTyr-G-OH (SEQ ID NO:2) were synthesized by Fmoc-based solid phase peptide synthesis and purified by HPLC. Each peptide was subjected to the following trypsin cleavage protocol:

Trypsin Activation: Trypsin (20 μg, sequencing grade modified trypsin, Promega Corporation, Madison, Wis., USA, catalogue no. V511, specific activity 16.965 u/mg) was dissolved in Trypsin resuspension buffer (200 μL, Promega, catalogue no. V542A, ingredient 50 mM acetic acid) and incubated for 15 min at 37° C.

Reduction and alkylation: The peptide (approx. 0.05 mg) was dissolved in 1 M urea and 100 mM fresh ammonium hydrogen carbonate (300 μL). TCEP (Tris(2-carboxyethyl) phosphine hydrochloride, 0.2 M, 7.5 μL, final concentration 5 mM) was added and the solution incubated at 37° C. for 1 h. After addition of iodoacetamide (0.5 M, 6.0 μL, final concentration 10 mM) the solution was incubated at RT in the dark for another 30 min. Digestion: The sample was digested with activated Trypsin solution (10 μL per well, 1.0 μg, resulting enzyme/substrate ratio 1:50) over night at RT. To stop the digestion, HCl (2 M, 8 μL final concentration 50 mM) and TFA (3.2 μL, final concentration 1%, pH<2) were added. Finally, the sample was analyzed by LC-MS at 350 nm.

Table 2 shows that the cleavage efficiency—as determined by LC-MS and UV detection at 350 nm—was >99% in all cases, with the exception of peptide 20 (HLVSPEALDLLD-K-SA-nitroTyr-G (SEQ ID NO:28)) where it was only 75%. This means that except for one peptide the cleavage of the quantification tag is basically quantitative.

Peptide 20 bears an aspartic acid attached to the C-terminal lysine. It is known that peptide bonds between Lys or Arg and the acidic amino acids Asp and Glu are often cleaved slowly by trypsin (Rehm, H.; Letzel, T. Der Experimentator Proteinbiochemie/Proteomics, 6^{th} edition, 2010, Spektrum Akademischer Verlag, Heidelberg, p. 242). However, the incomplete cleavage of these bonds is not problematic for the application of tagged peptides in MS-based proteomics, since this information can be taken into account for the selection process of proteotypic peptides. Practically this means that peptides with D-K- and E-K-bonds will simply not be chosen as proteotypic peptides for the detection and quantification of proteins.

The reduction and the alkylation process were applied to the test peptides, because it was aimed to subject the peptides to the same workflow as it is usually employed for the preparation of proteotypic peptides from proteins. This has the advantage, that the tagged peptides can be spiked into biological samples prior to their cleavage by tryptic digest, and thus are cleaved concomitantly with the protein. As all cleavage experiments led to the desired products, it can be concluded that the quantification tag did neither interfere with the reduction nor with the alkylation step.

TABLE 2
Cleavage of the C-terminal quantification tag-SA-nitroTyr-G (SEQ ID NO: 2)
from 23 different peptides.

| | | Percent | |
|---|---|---|---|
| | | Cleavage of | |
| Pept | | Tag [by UV | SEQ ID |
| # | Sequence | at 350 nm] | NO: |
| 1 | IISIFSGTE-K-SA-nitroTyr-G | 100 | 8 |
| 2 | SVIDPVPAPVGDSHVDGAA-K-SA-nitroTyr-G | 100 | 9 |
| 3 | LGINLLGGPLGG-K-SA-nitroTyr-G | 100 | 10 |
| 4 | SPEVLLGSA-R-SA-nitroTyr-G | 100 | 11 |
| 5 | NLDENGLDLLS-K-SA-nitroTyr-G | 100 | 12 |
| 6 | LDTETEGVPSTAI-R-SA-nitroTyr-G | 100 | 13 |
| 7 | LTGEVVAL-K-SA-nitroTyr-G | 100 | 14 |
| 8 | TVGALQVLGTEAQSSLL-K-SA-nitroTyr-G | 100 | 15 |
| 9 | NLSSNEAISLEEI-R-SA-nitroTyr-G | 99.2 | 16 |
| 10 | HGDLPDIQI-K-SA-nitroTyr-G | 100 | 17 |
| 11 | AALSALESFL-K-SA-nitroTyr-G | 100 | 18 |
| 12 | SLGPPQGEEDSVP-R-SA-nitroTyr-G | 100 | 19 |
| 13 | GQVFDVGP-R-SA-nitroTyr-G | 99.4 | 20 |
| 14 | ELIFEETA-R-SA-nitroTyr-G | 99.6 | 21 |
| 15 | LDLDLTADSQPPVF-K-SA-nitroTyr-G | 100 | 22 |
| 16 | LSPPYSSPQEFAQDVG-R-SA-nitroTyr-G | 100 | 23 |
| 17 | DHQYQFLEDAV-R-SA-nitroTyr-G | 100 | 24 |
| | LPNGVFTPDFQEFVN-K-SA-nitroTyr-G | 95.4 | 25 |
| 18 | LGLEDFESL-K-SA-nitroTyr-G | 100 | 26 |
| 19 | LIDWGLAEFYHPAQEYNV-R-SA-nitroTyr-G | 100 | 27 |
| 20 | HLVSPEALDLLD-K-SA-nitroTyr-G | 74.7 | 28 |
| 21 | EQSQPC(Acm)ADNAVLSSGLTAA-R-SA-nitroTyr-G | 100 | 29 |
| 22 | ELIFQETA-R-SA-nitroTyr-G | 100 | 30 |
| 23 | LAYTFEVQ-K-SA-nitroTyr-G | 100 | 31 |

### EXAMPLE 10

### Peptide Quantifications with a Nitro-Tyrosine-Based Tag: Efficiency of Tag Cleavage from 38 Different Peptides

38 peptides carrying the N-terminal quantification-tag Ac-nitroTyr-SGK-(SEQ ID NO:32) were synthesized by SPOT synthesis (Table 3). The peptide sequences are based on two different amino acid sequences, which in each case contain 19 different amino acids N-terminally attached to lysine (the 20 naturally occurring amino acids except cysteine).

All peptides were subjected to the same trypsin cleavage protocol as described in example 9. The cleavage efficiency for the 38 peptides—as determined by LC-MS and UV detection at 350 nm—is shown in table 3. It is apparent that the quantification tag is efficiently cleaved (>99%) in nearly all cases.

Four peptides were not efficiently cleaved. These are the peptides containing a K-D-(peptides 3 and 23) and a K-P-bond (peptides 13 and 33). This is not surprising, as it is known that peptide bonds between Lys/Arg and Pro as well as the acidic amino acids Asp and Glu are often cleaved slowly by trypsin (Rehm, H.; Letzel, T. Der Experimentator Proteinbiochemie/Proteomics, 6^{th} edition, 2010, Spektrum Akademischer Verlag, Heidelberg, p. 242). However, the incomplete cleavage of these bonds is not problematic for the application of tagged peptides in MS-based proteomics, since this information can be taken into account for the selection process of proteotypic peptides. Practically this means that peptides with D-K-, E-K- and P-K-bonds will simply not be chosen as proteotypic peptides for the detection and quantification of proteins.

Peptides with a KK- or KR-motif were cleaved C-terminally to this group with good cleavage efficiencies (peptides 9, 15, 29, 35).

TABLE 3
Cleavage of the N-terminal quantification tag Ac-nitroTyr-SGK- (SEQ ID NO: 32)
from 38 different peptides.

| | | Kusebauch 24 h, RT: | |
|---|---|---|---|
| | | Percent Cleavage of | |
| Peptide | Sequence | Tag [by UV350 - | SEQ ID |
| # | | Peptide-Tag vs. Tag] | NO: |
| 1 | Ac-nitroTyr-SGKATHYFIDLN-K-OH | 100.0 | 33 |
| 3 | Ac-nitroTyr-SGKDTHYFIDLN-K-OH | 60.2 | 34 |
| 4 | Ac-nitroTyr-SGKETHYFIDLN-K-OH | 100.0 | 35 |
| 5 | Ac-nitroTyr-SGKFTHYFIDLN-K-OH | 99.0 | 36 |
| 6 | Ac-nitroTyr-SGKGTHYFIDLN-K-OH | 100.0 | 37 |
| 7 | Ac-nitroTyr-SGKHTHYFIDLN-K-OH | 100.0 | 38 |
| 8 | Ac-nitroTyr-SGKITHYFIDLN-K-OH | 100.0 | 39 |
| 9 | Ac-nitroTyr-SGKKTHYFIDLN-K-OH | 100.0 | 40 |
| 10 | Ac-nitroTyr-SGKLTHYFIDLN-K-OH | 100.0 | 41 |
| 11 | Ac-nitroTyr-SGKMTHYFIDLN-K-OH | 100.0 | 42 |
| 12 | Ac-nitroTyr-SGKNTHYFIDLN-K-OH | 100.0 | 43 |
| 13 | Ac-nitroTyr-SGKPTHYFIDLN-K-OH | 37.0 | 44 |
| 14 | Ac-nitroTyr-SGKQTHYFIDLN-K-OH | 100.0 | 45 |
| 15 | Ac-nitroTyr-SGKRTHYFIDLN-K-OH | 100.0 | 46 |
| 16 | Ac-nitroTyr-SGKSTHYFIDLN-K-OH | 100.0 | 47 |
| 17 | Ac-nitroTyr-SGKTTHYFIDLN-K-OH | 100.0 | 48 |
| 18 | Ac-nitroTyr-SGKVTHYFIDLN-K-OH | 100.0 | 49 |
| 19 | Ac-nitroTyr-SGKWTHYFIDLN-K-OH | 100.0 | 50 |
| 20 | Ac-nitroTyr-SGKYTHYFIDLN-K-OH | 100.0 | 51 |
| 21 | Ac-nitroTyr-SGKAVALSLDDV-K-OH | 100.0 | 52 |
| 23 | Ac-nitroTyr-SGKDVALSLDDV-K-OH | 59.2 | 53 |
| 24 | Ac-nitroTyr-SGKEVALSLDDV-K-OH | 100.0 | 54 |
| 25 | Ac-nitroTyr-SGKFVALSLDDV-K-OH | 100.0 | 55 |
| 26 | Ac-nitroTyr-SGKGVALSLDDV-K-OH | 99.3 | 56 |
| 27 | Ac-nitroTyr-SGKHVALSLDDV-K-OH | 100.0 | 57 |
| 28 | Ac-nitroTyr-SGKIVALSLDDV-K-OH | 100.0 | 58 |
| 29 | Ac-nitroTyr-SGKKVALSLDDV-K-OH | 100.0 | 59 |
| 30 | Ac-nitroTyr-SGKLVALSLDDV-K-OH | 100.0 | 60 |
| 31 | Ac-nitroTyr-SGKMVALSLDDV-K-OH | 100.0 | 61 |
| 32 | Ac-nitroTyr-SGKNVALSLDDV-K-OH | 100.0 | 62 |
| 33 | Ac-nitroTyr-SGKPVALSLDDV-K-OH | 5.4 | 63 |
| 34 | Ac-nitroTyr-SGKQVALSLDDV-K-OH | 100.0 | 64 |
| 35 | Ac-nitroTyr-SGKRVALSLDDV-K-OH | 100.0 | 65 |
| 36 | Ac-nitroTyr-SGKSVALSLDDV-K-OH | 100.0 | 66 |
| 37 | Ac-nitroTyr-SGKTVALSLDDV-K-OH | 100.0 | 67 |
| 38 | Ac-nitroTyr-SGKVVALSLDDV-K-OH | 100.0 | 68 |
| 39 | Ac-nitroTyr-SGKWVALSLDDV-K-OH | 100.0 | 69 |
| 40 | Ac-nitroTyr-SGKYVALSLDDV-K-OH | 99.1 | 70 |

The features of the present invention disclosed in the specification, the claims, the sequence listing and/or the drawings may both separately and in any combination thereof be material for realizing the invention in various forms thereof.

1-90. (canceled)

91. A method for determining the presence and/or quantity of a target polypeptide of which a first peptide is a proteotypic peptide, in at least one mixture of different polypeptides, comprising:
a) providing a mixture of different polypeptides containing the target polypeptide;
b) adding a quantity of (i) an internal peptide standard comprising a tag to which is coupled a first peptide comprising an amino acid sequence, wherein the C-terminal end of the amino acid sequence of the first peptide corresponds to the C-terminal end generated by a sequence-specific hydrolysis of an amide bond, ester bond, or thioester bond of a peptide, or (ii) a plurality of different species of said internal peptide standard, said different species of said internal peptide standard differing from each other in the tag and/or in the amino acid sequence of the first peptide, thereby generating a spiked mixture;
c) generating a plurality of peptides from the mixture of different polypeptides and cleaving off the tag from the internal peptide standard/standards, thus releasing the first peptide, whereby the plurality of peptides comprises the proteotypic peptide derived from the different polypeptides and the first peptide released from the internal standard;
d) determining the amount of the tag contained in the spiked mixture and therefrom the quantity of the internal standard/standards added in step b);
e) determining the ratio of the first peptide to the proteotypic peptide derived from the different polypeptides, each generated in step c); and
f) calculating from the ratio and the quantity of the internal standard/standards determined in step d) the quantity of the target polypeptide in the mixture of different polypeptides,
wherein either the proteotypic peptide derived from the different polypeptides or the first peptide of the internal peptide standard/standards comprise a mass label.

92. The method according to claim 91, wherein the mass label is selected from the group consisting of C isotopes and N isotopes.

93. The method according to claim 91, wherein the ratio of the first peptide released from the internal peptide standard in step c) to the proteotypic peptide derived from the different polypeptides in step c) is determined by mass spectrometry.

94. (canceled)

95. The method according to claim 91, wherein a peptide signature obtained by fragmentation and subsequent analysis in a tandem mass spectrometer which is diagnostic for the presence of the peptide is known for the proteotypic peptide.

96. (canceled)

97. The method according to claim 91, wherein the internal peptide standard or the plurality of different species of said internal peptide standard are not purified prior to adding to the mixture of different polypeptides containing the target polypeptide.

98. The method according to claim 91, wherein the internal peptide standard or the plurality of different species of said internal peptide standard are purified prior to adding to the mixture of different polypeptides containing the target polypeptide.

99. (canceled)

100. The method according to claim 91, wherein the tag comprises a fluorescence label and the amount of the tag is determined by measuring the fluorescence signal of the label.

101. The method according to claim 91, wherein the tag comprises a UV label and the amount of the tag is determined by measuring the UV signal of the label.

102. The method according to claim 91, wherein after step c) and prior to step d) the treated spiked mixture is subjected to a separation step where the first tag is separated from components of the treated spiked mixture.

103. The method according to claim 91, wherein after step c) and prior to step e) the treated spiked mixture is subjected to a separation step where the first peptide and the proteotypic peptide derived from the different polypeptides are separated from components of the treated spiked mixture.

104. A method for determining the presence and/or quantity of a target polypeptide of which a first peptide is a proteotypic peptide, in at least one mixture of different polypeptides, comprising:
a) providing a mixture of different polypeptides preferably containing the target polypeptide;
b) providing (i) an internal peptide standard comprising a tag to which is coupled a first peptide comprising an amino acid sequence, wherein the C-terminal end of the amino acid sequence of the first peptide corresponds to the C-terminal end generated by a sequence-specific hydrolysis of an amide bond, ester bond or thioester bond of a peptide, or (ii) a plurality of different species of said internal peptide standard, said different species of said internal peptide standard differing from each other in the tag and/or in the amino acid sequence of the first peptide; and treating the internal peptide standard or the plurality of different species of said internal peptide standard with a means for sequence-specific hydrolysis to cleave off the tag from the internal peptide standard or the plurality of different species of said internal peptide standard, thus releasing the first peptide;
either
ca1) adding the reaction mixture obtained in step b) to the mixture of different polypeptides of step a); and
ca2) treating the mixture obtained in step ca1) with a means for sequence-specific hydrolysis to generate a plurality of peptides from the mixture of different polypeptides, whereby the plurality of peptides comprises the proteotypic peptide;
or
cb1) treating the mixture of different polypeptides of step a) with a means for sequence-specific hydrolysis to generate a plurality of peptides from the mixture of different polypeptides, whereby the plurality of peptides comprises the proteotypic peptide; and
cb2) adding the reaction mixture obtained in step b) to the mixture obtained in step cb1);
whereby after step ca2) and step cb2), respectively, a spiked mixture is obtained;
d) determining the amount of the first tag contained in the spiked mixture and therefrom the quantity of the internal standard/standards added in step b);
e) determining in the spiked mixture the ratio of the first peptide to the proteotypic peptide derived from the different polypeptides; and
f) calculating from the ratio and the quantity of the internal standard/standards determined in step d) the quantity of the target polypeptide in the mixture of different polypeptides,
wherein either the proteotypic peptide derived from the different polypeptides or the first peptide of the internal peptide standard/standards comprise a mass label.

105. The method according to claim 104, wherein the mass label is selected from the group consisting of C isotopes and N isotopes.

106. The method according to claim 104, wherein the ratio of the first peptide released from the internal peptide standard to the proteotypic peptide derived from the different polypeptides is determined by mass spectrometry.

107. (canceled)

108. The method according to claim 104, wherein a peptide signature obtained by fragmentation and subsequent analysis in a tandem mass spectrometer which is diagnostic for the presence of the peptide is known for the proteotypic peptide.

109. The method according to claim 104, wherein the means for sequence-specific hydrolysis is selected from a proteolytic enzyme, a sequence-specific chemical reaction or a sequence-specific physical treatment.

110. The method according to claim 104, wherein the internal peptide standard or the plurality of different species of said internal peptide standard are not purified prior to adding to the mixture of different polypeptides containing the target polypeptide.

111. The method according to claim 104, wherein the internal peptide standard or the plurality of different species of said internal peptide standard are purified prior to adding to the mixture of different polypeptides containing the target polypeptide.

112. (canceled)

113. The method according to claim 104, wherein the tag comprises a fluorescence label and the amount of the tag is determined by measuring the fluorescence signal of the label.

114. The method according to claim 104, wherein the tag comprises a UV label and the amount of the tag is determined by measuring the UV signal of the label.

115. (canceled)

116. The method according to claim 104, wherein prior to step e) the spiked mixture is subjected to a separation step where the first peptide and the proteotypic peptide derived from the different polypeptides are separated from components of the treated spiked mixture.

117. The method according to claim 104, wherein the means for sequence-specific hydrolysis used in step b) is removed from the mixture obtained in the performance of step b) prior to performing step ca1) or step cb1).

118. A method for determining the presence and/or quantity of a target polypeptide of which a first peptide is a proteotypic peptide, in at least one mixture of different polypeptides, comprising:
a) providing (i) an internal peptide standard comprising a tag to which is coupled a first peptide comprising an amino acid sequence, wherein the C-terminal end of the amino acid sequence of the first peptide corresponds to the C-terminal end generated by a sequence-specific hydrolysis of an amide bond, ester bond, or thioester bond of a peptide, or (ii) a plurality of different species of said internal peptide standard, said different species of said internal peptide standard differing from each other in the tag and/or in the amino acid sequence of the first peptide; and treating the internal peptide standard or the plurality of different species of said internal peptide standard with a means for sequence-specific hydrolysis to cleave off the tag from the internal peptide standard or the plurality of different species of said internal peptide standard, thus releasing the first peptide;
b) optionally removing the means for sequence-specific hydrolysis;
c) determining the amount of the first tag contained in the mixture obtained from step a) or step b) and therefrom the amount of the internal standard contained in said mixture;
d) providing a mixture of different polypeptides containing the target polypeptide;
either
da1) adding a part of the reaction mixture obtained in step a) or step b) to the mixture of different polypeptides of step d); and
da2) treating the mixture obtained in step da1) with a means for sequence-specific hydrolysis to generate a plurality of peptides from the mixture of different polypeptides, whereby the plurality of peptides comprises the proteotypic peptide;
or
db1) treating the mixture of different polypeptides of step d) with a means for sequence-specific hydrolysis to generate a plurality of peptides from the mixture of different polypeptides, whereby the plurality of peptides comprises the proteotypic peptide; and
db2) adding a part of the mixture obtained in step a) or step b) to the mixture obtained in step db1);
whereby after step da2) and step db2), respectively, a spiked mixture is obtained;
e) determining in the spiked mixture the ratio of the first peptide to the proteotypic peptide derived from the different polypeptides; and
f) calculating from the ratio and the quantity of the internal standard/standards the quantity of the target polypeptide in the mixture of different polypeptides,
wherein either the proteotypic peptide derived from the different polypeptides or the first peptide of the internal peptide standard/standards comprise a mass label.

119. The method according to claim 118, wherein the mass label is selected from the group consisting of C isotopes and N isotopes.

120. The method according to claim 118, wherein the ratio of the first peptide released from the internal peptide standard to the proteotypic peptide derived from the different polypeptides is determined by mass spectrometry.

121. (canceled)

122. The method according to claim 118, wherein a peptide signature obtained by fragmentation and subsequent analysis in a tandem mass spectrometer which is diagnostic for the presence of the peptide is known for the proteotypic peptide.

123. The method according to claim 118, wherein the means for sequence-specific hydrolysis is selected from a proteolytic enzyme, a sequence-specific chemical reaction or a sequence-specific physical treatment.

124. The method according to claim 118, wherein the internal peptide standard or the plurality of different species of said internal peptide standard are not purified prior to adding to the mixture of different polypeptides containing the target polypeptide.

125. The method according to claim 118, wherein the internal peptide standard or the plurality of different species of said internal peptide standard are purified prior to adding to the mixture of different polypeptides containing the target polypeptide.

126. (canceled)

127. The method according to claim 118, wherein the tag comprises a fluorescence label and the amount of the tag is determined by measuring the fluorescence signal of the label.

128. The method according to claim 118, wherein the tag comprises a UV label and the amount of the tag is determined by measuring the UV signal of the label.

129. The method according to claim 118, wherein
a) prior to step da1) the reaction mixture obtained in step a) or step b) is subjected to a separation step where the tag is separated from components of said reaction mixture; or
b) prior to step db2) the reaction mixture obtained in step db1) is subjected to a separation step where the proteotypic peptide derived from the different polypeptides is separated from components of said reaction mixture.
